(19) United States
(12) Patent Application Publication
(10) Pub. No.: US 2025/0177378 A1
(43) Pub. Date: Jun. 5, 2025

(54) TREATMENT OF DEPRESSION

(71) Applicant: ANTECIP BIOVENTURES II LLC, New York, NY (US)

(72) Inventor: Herriot Tabuteau, New York, NY (US)

(21) Appl. No.: 18/923,576

(22) Filed: Oct. 22, 2024

Related U.S. Application Data
(63) Continuation of application No. PCT/US2023/067062, filed on May 16, 2023.
(60) Provisional application No. 63/342,960, filed on May 17, 2022.
(60) Provisional application No. 63/345,396, filed on May 24, 2022.
(60) Provisional application No. 63/345,856, filed on May 25, 2022.
(60) Provisional application No. 63/346,083, filed on May 26, 2022.
(60) Provisional application No. 63/346,269, filed on May 26, 2022.
(60) Provisional application No. 63/347,431, filed on May 31, 2022.

(51) Int. Cl. A61K 31/485; A61K 9/00; A61K 31/137; A61P 25/22; A61P 25/24

(52) U.S. Cl. CPC A61K 31/485; A61K 9/0053; A61K 31/137; A61P 25/22; A61P 25/24

(57) ABSTRACT

This disclosure relates to administration of a combination of: 1) about 100-110 mg, about 104-106 mg, or about 105 mg of bupropion hydrochloride, or a molar equivalent amount of the free base form or another salt form of bupropion; and 2) about 40-50 mg, about 44-46 mg, or about 45 mg of dextromethorphan hydrobromide, or a molar equivalent amount of the free base form or another salt form of dextromethorphan in certain patient populations for treating pain or a neurological disorder such as depression.

### CROSS-REFERENCE TO RELATED APPLICATIONS

This application is a continuation of International Pat. App. No. PCT/US2023/067062, filed May 16, 2023; which claims the benefit of U.S. Provisional App. Nos. 63/342,960, filed May 17, 2022; 63/345,396, filed May 24, 2022; 63/345,856, filed May 25, 2022; 63/346,083, filed May 26, 2022; 63/346,269, filed May 26, 2022; and 63/347,431, filed May 31, 2022; all of which are incorporated by reference herein in their entireties.

### SUMMARY

This disclosure relates to administration of a combination of: 1) about 100-110 mg, about 104-106 mg, or about 105 mg of bupropion hydrochloride, or a molar equivalent amount of the free base form or another salt form of bupropion; and 2) about 40-50 mg, about 44-46 mg, or about 45 mg of dextromethorphan hydrobromide, or a molar equivalent amount of the free base form or another salt form of dextromethorphan in certain patient populations for treating pain or a neurological disorder such as depression.

### BRIEF DESCRIPTION OF THE DRAWINGS

FIG. 1 depicts the flow chart of participants through a phase 2 trial (a randomized double-blind controlled trial) of dextromethorphan-bupropion for major depressive disorder in Example 1.

FIG. 2A displays the mean change from baseline in the MADRS total score over time with Dextromethorphan-Bupropion compared with bupropion alone in Example 1. Error bars indicate standard error, and p values are calculated on least squares means. MADRS=Montgomery-Åsberg Depression Rating Scale.

FIG. 2B displays the percentage of patients achieving remission (with MADRS total score≤10) over time in week with Dextromethorphan-Bupropion compared with bupropion alone in Example 1. P values are calculated on least squares means. MADRS=Montgomery-Åsberg Depression Rating Scale.

FIG. 3 depicts the study procedure in Example 3.

FIG. 4 depicts the time to relapse in weeks Dextromethorphan-Bupropion compared with placebo in Example 3.

FIG. 5 displays a plot of mean reduction in MADRS total score over time in month with Dextromethorphan-Bupropion treatment in Example 4.

FIG. 6 displays plots of proportion of subjects in percentage had clinical response (with MADRS reduced ≥50%) and remission (with MADRS≤10) over time in week and month with Dextromethorphan-Bupropion treatment in Example 4.

FIG. 7 displays plots of proportion of subjects in percentage had CGI-S response (≥2 category change) over time in week and month with Dextromethorphan-Bupropion treatment in Example 4.

FIG. 8 displays the mean reduction in SDS over time in month with Dextromethorphan-Bupropion in Example 4. Error bars indicate standard error.

FIG. 9 displays the proportion of subjects (in percentage) in SDS remission (SDS≤6) over time in week and month with Dextromethorphan-Bupropion treatment in Example 4.

FIG. 10 displays the mean reduction in Hamilton Anxiety Rating Scale (HAM-A) total scores over time in month with Dextromethorphan-Bupropion treatment in Example 5.

FIG. 11 displays the percentage of patients achieving remissionon on the HAM-A (score≤7) over time in week and month with Dextromethorphan-Bupropion treatment in Example 5.

FIG. 12 displays the percent of patients achieving HAM-A response (≥50% improvement from Baseline) over time in week and month with Dextromethorphan-Bupropion treatment in Example 5.

FIG. 13 depicts the study design for GEMINI, a phase 3, randomized, double-blind, placebo-controlled, multi-center, U.S. trial with either Dextromethorphan-Bupropion or placebo (NCT04019704) in Example 6.

FIG. 14 displays the MADRS total score mean change from baseline over time in week with Dextromethorphan-Bupropion compared to placebo in Example 6. P-Values were calculated on LS means.

FIG. 15 displays the MADRS Anhedonia subscale LS mean change over time in week with Dextromethorphan-Bupropion compared to placebo in Example 6.

FIG. 16 displays the percentage of responders achieving ≥50% Reduction in MADRS Anhedonia Subscale over time in week with Dextromethorphan-Bupropion compared to placebo in Example 6.

FIG. 17 depicts the patient disposition in a phase 3 trial of Dextromethorphan-Bupropion for major depressive disorder in Example 7.

FIG. 18A displays the MADRS total score mean change from baseline over time in week with Dextromethorphan-Bupropion compared to placebo in Example 7. Scores on MADRS range from 0 to 60, with higher scores indicating more severe depression. The results are presented as means, and the error bars represent standard errors. P values are calculated based on least-square means. MADRS=Montgomery-Åsberg depression Rating Scale, SE=standard error.

FIG. 18B displays the percentage of patients achieving remission (MADRS total score≤10) over time in week with Dextromethorphan-Bupropion compared to placebo in Example 7. P values are calculated via X² tests. MADRS=Montgomery-Åsberg depression Rating Scale.

FIG. 18C displays the percentage of patients achieving clinical response (≥50% reduction in MADRS total score from baseline) over time in week with Dextromethorphan-Bupropion compared to placebo in Example 7. P values are calculated via X² tests. MADRS=Montgomery-Åsberg depression Rating Scale.

### DETAILED DESCRIPTION

As mentioned above, this disclosure relates to administration of a combination of: 1) about 100-110 mg, about 104-106 mg, or about 105 mg of bupropion hydrochloride, or a molar equivalent amount of the free base form or another salt form of dextromethorphan; and 2) about 40-50 mg, about 44-46 mg, or about 45 mg of dextromethorphan hydrobromide, or a molar equivalent amount of the free base form or another salt form of dextromethorphan. This combination is referred to for convenience herein as the “subject combination.” In every instance where the subject combination is referred to herein, the combination of 105 mg of bupropion hydrochloride and 45 mg of dextromethorphan hydrobromide is specifically contemplated.

Dextromethorphan hydrobromide is an uncompetitive NMDA receptor antagonist and A sigma-1 receptor agonist.

The chemical name of dextromethorphan hydrobromide is morphinan, 3-methoxy-17-methyl-, (9α, 13α, 14α), hydrobromide monohydrate. Dextromethorphan hydrobromide has the empirical formula C₁₈H₂₅NO·HBr·H₂O and a molecular weight of 370.33. The structural formula is:

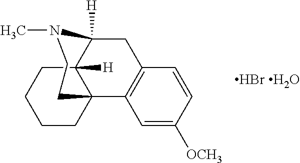 [SMILES: COc1ccc2c(c1)[C@]13CCCC[C@@H]1[C@H](C2)N(C)CC3]

Dextromethorphan hydrobromide powder is white or almost white, crystalline, and sparingly soluble in water.

Bupropion hydrochloride is an aminoketone and CYP450 2D6 inhibitor.

The chemical name of bupropion hydrochloride is: (±)-1-(3-chlorophenyl)-2-[(1,1-dimethylethyl)amino]-1-propanone hydrochloride. Bupropion hydrochloride has the empirical formula C₁₃H₁₈ClNO·HCl and a molecular weight of 276.2. The structural formula is:

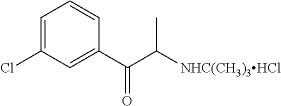

Bupropion hydrochloride powder is white and highly soluble in water.

The subject combination may be contained in an oral dosage form, including a tablet, such as an extended-release tablet. In some embodiments, the subject combination is contained in a dosage form for oral administration and is available as round bilayer tablets or round single layer tablets.

In some embodiments, each tablet containing the subject combination contains 45 mg of dextromethorphan hydrobromide in an immediate-release formulation. In some embodiments, each tablet of the subject combination contains 105 mg of bupropion hydrochloride in an extended-release formulation. In some embodiments, each tablet of the subject combination contains 45 mg of dextromethorphan hydrobromide in an immediate-release formulation and 105 mg of bupropion hydrochloride in an extended-release formulation.

In some embodiments, a tablet containing the subject combination contains L-cysteine hydrochloride monohydrate. In some embodiments, a tablet containing the subject combination contains carbomer homopolymer. In some embodiments, a tablet containing the subject combination contains microcrystalline cellulose. In some embodiments, a tablet containing the subject combination contains colloidal silicon dioxide. In some embodiments, a tablet containing the subject combination contains crospovidone. In some embodiments, a tablet containing the subject combination contains stearic acid. In some embodiments, a tablet containing the subject combination contains magnesium stearate.

In some embodiments, a tablet containing the subject combination contains the following inactive ingredients: L-cysteine hydrochloride monohydrate, carbomer homopolymer, microcrystalline cellulose, colloidal silicon dioxide, crospovidone, stearic acid, and magnesium stearate.

In some embodiments, the starting dosage of the subject combination is 45 mg of dextromethorphan hydrobromide and 105 mg of bupropion hydrochloride in one tablet that is administered once daily in the morning. In some embodiments, after 3 days, the dosage is increased to one tablet (or one dosage form containing 45 mg of dextromethorphan hydrobromide and 105 mg of bupropion hydrochloride) twice daily, e.g., given at least 8 hours apart. In some embodiments, no more than two doses containing 45 mg of dextromethorphan hydrobromide and 105 mg of bupropion hydrochloride are administered in the same day.

The subject combination may be administered orally with or without food. In some embodiments, the tablets are swallowed whole, and not crushed, divided, or chewed.

In the subject combination, bupropion inhibits the metabolism of dextromethorphan via CYP2D6. Dextromethorphan, when co-administered with bupropion, displays nonlinear pharmacokinetics at steady state, with greater than dose-proportional changes in AUC and Cₘₐₓ of dextromethorphan for varying doses of dextromethorphan (30 mg to 60 mg) and less than dose-proportional changes in AUC and Cₘₐₓ of bupropion for varying doses of bupropion (75 mg to 150 mg).

Steady state plasma concentrations of dextromethorphan and bupropion when given as the subject combination are achieved within 8 days. The accumulation ratios for dextromethorphan at steady state are about 20 and about 32, respectively based on Cₘₐₓ and AUC₀₋₁₂. The accumulation ratios for bupropion at steady state are 1.1 and 1.5, respectively based on Cₘₐₓ and AUC₀₋₁₂.

After administration of the subject combination, the median Tₘₐₓ of dextromethorphan is about 3 hours and the median Tₘₐₓ of bupropion is about 2 hours. The Cₘₐₓ of hydroxybupropion metabolite occurs approximately 3 hours post-dose and is approximately 14 times the peak level of bupropion. The AUC₀₋₁₂ hydroxybupropion is about 19 times that of bupropion. The Cₘₐₓ of the erythrohydroxybupropion and threohydroxybupropion metabolites occurs approximately 4 hours post-dose and is approximately equal to and about 5 times that of bupropion, respectively. The AUC₀₋₁₂ values of erythrohydroxybupropion and threohydroxybupropion are about 1.2 and about 7 times that of bupropion, respectively.

The subject combination can be taken with or without food. Dextromethorphan Cₘₐₓ and AUC₀₋₁₂ were unchanged and decreased by 14%, respectively, and bupropion Cₘₐₓ and AUC₀₋₁₂ were increased by 3% and 6%, respectively, when the subject combination was administered with food.

The plasma protein binding of dextromethorphan is approximately 60-70% and the plasma protein binding of bupropion is 84%. The extent of protein binding of the hydroxybupropion metabolite is similar to that for bupropion; whereas the extent of protein binding of the threohydroxybupropion metabolite is about half that seen with bupropion.

Following 8 consecutive days of administration of the subject combination in extensive metabolizers, the mean elimination half-life of dextromethorphan was increased approximately 3-fold to about 22 hours, as compared to dextromethorphan given without bupropion.

The mean elimination half-life of dextromethorphan and bupropion was 22 hours and 15 hours, respectively. The apparent elimination half-life of hydroxybupropion, erythrohydroxybupropion and threohydroxybupropion metabolites were approximately 35, 44 and 33 hours, respectively.

The subject combination may be used for adjunctive treatment of major depressive disorder or depression.

In addition to major depressive disorder, the subject combination may be used to treat other diseases in conditions in the patient populations or circumstances described herein. For example, the subject combination may be used to treat pain or a neurological disorder. Examples of neurological disorders that may be treated with the subject combination include, but are not limited to: affective disorders, psychiatric disorders, cerebral function disorders, movement disorders, dementias, motor neuron diseases, neurodegenerative diseases, seizure disorders, and headaches.

Affective disorders that may be treated by the subject combination include, but are not limited to, depression, major depression, treatment resistant depression, treatment resistant bipolar depression, bipolar disorders including cyclothymia, seasonal affective disorder, mood disorders, chronic depression (dysthymia), psychotic depression, postpartum depression, premenstrual dysphoric disorder (PMDD), situational depression, atypical depression, mania, anxiety disorders, attention deficit disorder (ADD), attention deficit disorder with hyperactivity (ADDH), and attention deficit/hyperactivity disorder (AD/HD), bipolar and manic conditions, obsessive-compulsive disorder, bulimia, obesity or weight-gain, narcolepsy, chronic fatigue syndrome, premenstrual syndrome, substance addiction or abuse, nicotine addiction, psycho-sexual dysfunction, pseudobulbar affect, and emotional lability.

Depression may be manifested by depressive symptoms. These symptoms may include psychological changes such as changes in mood, feelings of intense sadness, despair, mental slowing, loss of concentration, pessimistic worry, agitation, anxiety, irritability, guilt, anger, feelings of worthlessness, reckless behavior, suicidal thoughts, or attempts, and/or self-deprecation. Physical symptoms of depression may include insomnia, anorexia, appetite loss, weight loss, weight gain, decreased energy and libido, fatigue, restlessness, aches, pains, headaches, cramps, digestive issues, and/or abnormal hormonal circadian rhythms.

Psychiatric disorders that may be treated by the subject combination, include, but are not limited to, anxiety disorders, including but not limited to, phobias, generalized anxiety disorder, social anxiety disorder, panic disorder, agoraphobia, obsessive-compulsive disorder, and post-traumatic stress disorder (PTSD); mania, manic depressive illness, hypomania, unipolar depression, depression, stress disorders, somatoform disorders, personality disorders, psychosis, schizophrenia, delusional disorder, schizoaffective disorder, schizotypy, aggression, aggression in Alzheimer's disease, agitation, and agitation in Alzheimer's disease. Alzheimer's disease may also be referred to as dementia of the Alzheimer's type. Other neurobehavioral symptoms of Alzheimer's disease that may be treated include disinhibition and apathy.

Agitation in Alzheimer's disease occurs as the disease progresses. Agitation may present itself as inappropriate verbal, emotional, and/or physical behaviors. Inappropriate behaviors may include, but are not limited to, incoherent babbling, inappropriate emotional response, demands for attention, threats, irritability, frustration, screaming, repetitive questions, mood swings, cursing, abusive language, physical outbursts, emotional distress, restlessness, shredding, sleeping disturbances, delusions, hallucinations, pacing, wandering, searching, rummaging, repetitive body motions, hoarding, shadowing, hitting, scratching, biting, combativeness, hyperactivity, and/or kicking.

Alzheimer's disease (AD) is a progressive neurodegenerative disorder characterized by cognitive decline, and behavioral and psychological symptoms including agitation. AD is the most common form of dementia and afflicts an estimated 6 million individuals in the United States, a number that is anticipated to increase to approximately 14 million by 2050. Agitation is reported in up to 70% of patients with AD and is characterized by emotional distress, aggressive behaviors, disruptive irritability, and disinhibition. Managing agitation is a priority in AD. Agitation in patients with AD has been associated with increased caregiver burden, decreased functioning, accelerated cognitive decline, earlier nursing home placement, and increased mortality. There are currently no therapies approved by the FDA for the treatment of agitation in patients with AD.

Neurobehavioral symptoms have been known to appear during dementia and may be treated by the subject combination. Caregivers or families may feel more overwhelmed by patients' behavioral/psychological symptoms than by their cognitive impairment. Common forms of the syndrome are Alzheimer's disease, vascular dementia, dementia with Lewy bodies (abnormal aggregates of protein that develop inside nerve cells), and a group of diseases that contribute to frontotemporal dementia (degeneration of the frontal lobe of the brain). The symptoms that dementia patients have are similar to those of psychiatric disorders, but some are slightly different from each other. Neurobehavioral symptoms associated with dementia include depression, apathy, agitation, disinhibition, hallucinations, delusions, psychosis, impulsiveness, aggressiveness, compulsion, excessive sex drive, and personality disorders. Neurobehavioral symptoms such as disinhibition may also be found in other conditions such as traumatic brain injury.

Agitation in patients with Alzheimer's disease may be assessed using the Cohen Mansfield Agitation Inventory or CMAI. The CMAI assesses various behaviors including, Hitting (including self), Kicking, Grabbing onto people, Pushing, Throwing things, Biting, Scratching, Spitting, Hurting self or others, Tearing things or destroying property, Making physical sexual advances, Pacing, aimless wandering, Inappropriate dress or disrobing, Trying to get to a different place, Intentional falling, Eating/drinking inappropriate substances, Handling things inappropriately, Hiding things, Hoarding things, Performing repetitive mannerisms, General restlessness, Screaming, Making verbal sexual advances, Cursing or verbal aggression, Repetitive sentences or questions, Strange noises (weird laughter or crying), Complaining, Negativism, Constant unwarranted request for attention or help.

Schizophrenia may be treated by the subject combination including positive symptoms and/or negative symptoms of schizophrenia, or residual symptoms of schizophrenia. Other conditions that may be treated include intermittent explosive disorder.

Cerebral function disorders that may be treated by the subject combination include, but are not limited to, disorders involving intellectual deficits such as senile dementia, Alzheimer's type dementia, memory loss, amnesia/amnestic syndrome, epilepsy, disturbances of consciousness, coma, lowering of attention, speech disorders, voice spasms, Parkinson's disease, Lennox-Gastaut syndrome, autism, hyperkinetic syndrome, and schizophrenia. Cerebral function disorders also include disorders caused by cerebrovascular diseases including, but not limited to, stroke, cerebral infarction, cerebral bleeding, cerebral arteriosclerosis, cerebral venous thrombosis, head injuries, and the like where symptoms include disturbance of consciousness, senile dementia, coma, lowering of attention, and speech disorders.

Substance addiction abuse that may be treated by the subject combination includes, but is not limited to, drug dependence, addiction to cocaine, psychostimulants (e.g., crack, cocaine, speed, meth), nicotine, alcohol, opioids, anxiolytic and hypnotic drugs, cannabis (marijuana), amphetamines, hallucinogens, phencyclidine, volatile solvents, and volatile nitrites. Nicotine addiction includes nicotine addiction of all known forms, such as smoking cigarettes, cigars and/or pipes, e-cigarettes or vaping, and addiction to chewing tobacco.

Movement disorders that may be treated by the subject combination include, but are not limited to, akathisia, akinesia, associated movements, athetosis, ataxia, ballismus, hemiballismus, bradykinesia, cerebral palsy, chorea, Huntington's disease, Huntington's disease chorea, rheumatic chorea, Sydenham's chorea, dyskinesia, tardive dyskinesia, dystonia, blepharospasm, spasmodic torticollis, dopamine-responsive dystonia, Parkinson's disease, restless legs syndrome (RLS), tremor, essential tremor, and Tourette's syndrome, and Wilson's disease.

Dementias that may be treated by the subject combination include, but are not limited to, Alzheimer's disease, Parkinson's disease, vascular dementia, dementia with Lewy bodies, mixed dementia, fronto-temporal dementia, Creutzfeldt-Jakob disease, normal pressure hydrocephalus, Huntington's disease, Wernicke-Korsakoff Syndrome, and Pick's disease.

Motor neuron diseases that may be treated by the subject combination include, but are not limited to, amyotrophic lateral sclerosis (ALS), progressive bulbar palsy, primary lateral sclerosis (PLS), progressive muscular atrophy, post-polio syndrome (PPS), spinal muscular atrophy (SMA), spinal motor atrophies, Tay-Sach's disease, Sandhoff disease, and hereditary spastic paraplegia.

Neurodegenerative diseases that may be treated the subject combination include, but are not limited to, Alzheimer's disease, prion-related diseases, cerebellar ataxia, spinocerebellar ataxia (SCA), spinal muscular atrophy (SMA), bulbar muscular atrophy, Friedrich's ataxia, Huntington's disease, Lewy body disease, Parkinson's disease, amyotrophic lateral sclerosis (ALS or Lou Gehrig's disease), multiple sclerosis (MS), multiple system atrophy, Shy-Drager syndrome, corticobasal degeneration, progressive supranuclear palsy, Wilson's disease, Menkes disease, adrenoleukodystrophy, cerebral autosomal dominant arteriopathy with subcortical infarcts and leukoencephalopathy (CADASIL), muscular dystrophies, Charcot-Marie-Tooth disease (CMT), familial spastic paraparesis, neurofibromatosis, olivopontine cerebellar atrophy or degeneration, striatonigral degeneration, Guillain-Barré syndrome, and spastic paraplesia.

Seizure disorders that may be treated by the subject combination include, but are not limited to, epileptic seizures, nonepileptic seizures, epilepsy, febrile seizures; partial seizures including, but not limited to, simple partial seizures, Jacksonian seizures, complex partial seizures, and epilepsia partialis continua; generalized seizures including, but not limited to, generalized tonic-clonic seizures, absence seizures, atonic seizures, myoclonic seizures, juvenile myoclonic seizures, and infantile spasms; and status epilepticus.

Types of headaches that may be treated by the subject combination include, but are not limited to, migraine, tension, and cluster headaches.

Other neurological disorders that may be treated by the subject combination include, but are not limited to, Rett Syndrome, autism, tinnitus, disturbances of consciousness disorders, sexual dysfunction, intractable coughing, narcolepsy, cataplexy; voice disorders due to uncontrolled laryngeal muscle spasms, including, but not limited to, abductor spasmodic dysphonia, adductor spasmodic dysphonia, muscular tension dysphonia, and vocal tremor; diabetic neuropathy, chemotherapy-induced neurotoxicity, such as methotrexate neurotoxicity; incontinence including, but not limited, stress urinary incontinence, urge urinary incontinence, and fecal incontinence; and erectile dysfunction.

In some embodiments, the subject combination may be used to treat pain, joint pain, pain associated with sickle cell disease, pseudobulbar affect, depression (including treatment resistant depression), disorders related to memory and cognition, schizophrenia, Parkinson's disease, amyotrophic lateral sclerosis (ALS), Rhett's syndrome, seizures, cough (including chronic cough), etc.

In some embodiments, the subject combination may be administered orally to relieve musculoskeletal pain including low back pain, and pain associated with rheumatoid arthritis, juvenile rheumatoid arthritis, osteoarthritis, erosive osteoarthritis, sero-negative (non-rheumatoid) arthropathies, non-articular rheumatism, peri-articular disorders, axial spondyloarthritis including ankylosing spondylitis, Paget's disease, fibrous dysplasia, SAPHO syndrome, transient osteoarthritis of the hip, vertebral crush fractures, osteoporosis, etc.

In some embodiments, the subject combination may be administered to relieve inflammatory pain including musculoskeletal pain, arthritis pain, and complex regional pain syndrome.

Arthritis refers to inflammatory joint diseases that can be associated with pain. Examples of arthritis pain include pain associated with osteoarthritis, erosive osteoarthritis, rheumatoid arthritis, juvenile rheumatoid arthritis, sero-negative (non-rheumatoid) arthropathies, non-articular rheumatism, peri-articular disorders, neuropathic arthropathies including Charcot's foot, axial spondyloarthritis including ankylosing spondylitis, and SAPHO syndrome.

In some embodiments, the subject combination is used to treat chronic musculoskeletal pain.

In some embodiments, the subject composition may be administered to relieve complex regional pain syndrome, such as complex regional pain syndrome type I (CRPS-I), complex regional pain syndrome type II (CRPS-II), CRPS-NOS, or another type of CRPS. CRPS is a type of inflammatory pain. CRPS can also have a neuropathic component. Complex regional pain syndrome is a debilitating pain syndrome. It is characterized by severe pain in a limb that can be accompanied by edema, and autonomic, motor, and sensory changes.

In some embodiments, the subject composition may be administered orally to relieve neuropathic pain.

Examples of neuropathic pain include pain due to diabetic peripheral neuropathy or diabetic peripheral neuropathic pain, post-herpetic neuralgia, trigeminal neuralgia, monoradiculopathies, phantom limb pain, central pain, pain due to multiple sclerosis, etc. Other causes of neuropathic pain include cancer-related pain, lumbar nerve root compression, spinal cord injury, post-stroke pain, central multiple sclerosis pain, HIV-associated neuropathy, and radio- or chemo-therapy associated neuropathy, etc.

In some embodiments, the subject composition may be administered to relieve fibromyalgia.

In some embodiments, the subject combination may prevent relapse in patients with treatment-resistant depression (TRD). In general, about 40% of patients with MDD are treatment resistant, defined as non-response to two or more antidepressants correctly dosed from 2 different classes within the current depressive episode. Relapse of MDD is not uncommon in patients with TRD who eventually achieve remission, with >40% of patients relapsing within ˜4 months of treatment. Relapse was defined by ≥1 of the following: (1) MADRS total score ≥18 for 2 consecutive assessments, separated by 7 to 21 days; (2) ≥2-point increase from randomization on the Clinical Global Impression of Severity (CGI-S), with a minimum CGI-S score of 4, for 2 consecutive assessments, separated by 7 to 21 days; and (3) Loss of therapeutic response, such as: a) Hospitalization due to worsening of depression or due to risk of suicide, b) Investigator-determined need for additional antidepressant treatment or treatment switch, and c) Patient had relapsed in the opinion of the investigator.

In some embodiments, the subject combination may delay time to relapse of depressive symptoms compared to placebo in treatment resistant depression. In some embodiments, the subject combination may substantially and statistically delay time to relapse of depressive symptoms compared to placebo in TRD patients who have achieved stable remission on the subject combination (of dextromethorphan and bupropion).

In some embodiments, the patients with TRD continuing the treatment with the subject combination may not experience relapse of depressive symptoms within about 10 weeks, within about 20 weeks, within about 26 weeks, within about 30 weeks, within about 40 weeks, within about 50 weeks, within about 52 weeks or longer, or over at least 6 months of treatment.

In some embodiments, the subject combination may significantly prevent relapse of depression over at least 6 months compared to placebo. In some embodiments, treatment with the subject combination reduces the likelihood of relapse as compared to a placebo. In some embodiments, the likelihood that a patient will not experience relapse of depression with the treatment of the subject combination is at least 30%, about 30-40%, about 40-50%, or about 36%, or any percentage in the range bounded by any of the above values more than the percentage of patients without relapse of depression for receiving a placebo.

Depressive symptoms may be improved rapidly after treatment with the subject combination. The mean change in MADRS total score from a baseline may be significant at Week 1, Week 2, Week 4, or Week 6. The improvement in depressive symptomatology can last through about 3 months, about 6 months, about 1 year or longer. Clinical response (≥50% improvement) with the subject combination may be achieved at Week 1, Week 2, Week 4, or Week 6. Remission on the MADRS (≤10) with the subject combination may be achieved at Weeks 1, Week 2, Week 4, or Week 6 or longer. The antidepressant effects of the subject combination may be durable and sustained through Month 2, Month 3, Month 6, Month 9, or Month 12 or longer.

The response (≥2 category change) on the CGI-S with the treatment of the subject combination may be rapid. The response (≥2 category change) on the CGI-S may be observed at Week 1, Week 2, Week 4, or Week 6. The CGI-S improvement may be durable through Month 2, Month 3, Month 6, Month 9, or Month 12 or longer.

The mean reduction in SDS with the treatment of the subject combination may be rapid. The mean reduction in SDS with the treatment of the subject combination may be significant at Week 1, Week 2, Week 4, or Week 6. The mean reduction in SDS with the treatment of the subject combination may be durable and sustained through Month 2, Month 3, Month 6, Month 9, or Month 12 or longer. Remission on the SDS (≤6) may be achieved at Week 1, Week 2, Week 4, Week 6, Month 2, Month 3, Month 6, Month 9, or Month 12 or longer.

In some embodiments, the human patient is selected for having a HAM-A score of at least 10 or at least 15 prior to treatment.

Treatment with the subject combination may result in improvement in symptoms of anxiety. The reduction of the mean HAM-A scores from baseline may be significant at Week 1, Week 2, Week 4, or Week 6. The Improvements on the HAM-A may be durable through Month 2, Month 3, Month 6, Month 9, or Month 12 or longer. Treatment with the subject combination may result in rapid rates of remission of anxiety, at Week 1, Week 2, Week 4 or Week 6. The remission may be durable through Month 2, Month 3, Month 6, Month 9, or Month 12 or longer.

Response on the HAM-A (with ≥50% improvement from Baseline) may be achieved by about 15-70% of patients at Week 1, Week 2, Week 4, or Week 6, respectively. Response rates may continue to improve through Month 2, Month 3, Month 6, Month 9, or Month 12 or longer.

In some embodiments, the human patient is selected for having a MADRS Anhedonia score of at least 15 or at least 19 prior to treatment. In some embodiments, the subject composition may improve Anhedonic Symptoms in Major Depressive Disorder (MDD) as assessed by the MADRS anhedonia subscale. Anhedonia is one of the core features of MDD. Anhedonia is considered among the most bothersome aspects of MDD by patients, and has been associated with decreased functioning and is a risk factor for non-response to antidepressant therapy. MADRS Anhedonia Subscale includes 5-items: 1) apparent sadness; 2) reported sadness; 3) concentration difficulties; 4) lassitude; and 5) inability to feel.

In some embodiments, the subject combination may be administered orally to reduce the MADRS Anhedonia subscale at Week 1, Week 2, Week 3, Week 4, Week 5, or Week 6 or longer. In some embodiments, administering the subject combination may result in a mean reduction from baseline in the MADRS anhedonia subscale score of about 4-5, 4-6, 6-8, 6-7, 8-10, 8-9, 9-10, about 4.4, about 6.8, about 8.1, about 9.6, about 9.7, or about 10 or more, or any MADRS anhedonia subscale score in the range bounded by any of these values.

In some embodiments, the mean reduction from baseline in the MADRS anhedonia subscale for orally administering the subject combination may be significantly more than orally administering a placebo at Week 1, Week 2, Week 3, Week 4, Week 5, or Week 6 or longer. In some embodiments, the mean reduction from baseline in the MADRS anhedonia subscale for orally administering the subject combination may be about 30%, about 50%, about 60%, about 20-30%, about 30%-35%, about 30%-50%, about 40%-50%, about 50%-55%, about 50-60%, about 60%-65%, or any percentage in the range bounded by any of these values more than orally administering a placebo at week 1, week 2, week 3, week 4, week 5, or week 6 or longer.

In some embodiments, the likelihood that patient will be a responder (with ≥50% Reduction in MADRS Anhedonia Subscale) while being orally administered the subject combination may be about 20-30%, about 30-40%, about 40-50%, about 50-60%, about 20-25%, about 30-35%, about 40-45%, about 50-55%, about 21%, about 35%, about 42%, about 51%, about 54%, or any likelihood in the range bounded by any of these values at week 1, week 2, week 3, week 4, week 5, or week 6 or longer.

In some embodiments, the likelihood that patient will be a responder (with ≥50% Reduction in MADRS Anhedonia Subscale) may be statistically significantly greater for the subject combination of dextromethorphan and bupropion compared to a placebo at Week 1, Week 2, Week 3, Week 4, Week 5, or Week 6 or longer. In some embodiments, the likelihood that patient will be a responder while being administered the subject combination may be about 10-15%, about 15-20%, about 20-25%, about 13%, about 16%, about 18%, about 20%, about 23%, or any percentage in the range bounded by any of these values greater than the likelihood that a patient will be a responder while taking a placebo at Week 1, Week 2, Week 3, Week 4, Week 5, or Week 6 or longer.

The term “treating” or “treatment” includes the diagnosis, cure, mitigation, treatment, or prevention of disease in man or other animals, or any activity that otherwise affects the structure or any function of the body of man or other animals.

### Example 1

### Effect of Dextromethorphan-Bupropion in Major Depressive Disorder: A Randomized Double-Blind Controlled Trial

### Objective:

Altered glutamatergic neurotransmission is implicated in the pathogenesis of major depressive disorder. Dextromethorphan-bupropion is an oral NMDA receptor antagonist and sigma-1 receptor agonist, which utilizes inhibition of CYP2D6 to increase its bioavailability. This phase 2 trial assessed the efficacy and safety of dextromethorphan-bupropion in the treatment of major depressive disorder.

### Methods:

This randomized, double-blind, multicenter, parallel-group trial evaluated dextromethorphan-bupropion versus the active comparator sustained-release bupropion in patients 18-65 years old with a diagnosis of major depressive disorder of moderate or greater severity. Patients were randomly assigned to receive either dextromethorphan-bupropion (45 mg/105 mg tablet) or bupropion (105 mg tablet), once daily for the first 3 days and twice daily thereafter, for a total of 6 weeks. The primary endpoint was overall treatment effect on Montgomery-Åsberg Depression Rating Scale (MADRS) score (average of the change from baseline for weeks 1-6), assessed in all randomized patients whose diagnosis and severity were confirmed by an independent assessor and who received at least one dose of study medication and had at least one postbaseline assessment.

### Results:

Of 97 patients randomized, 17 did not have a confirmed diagnosis and severity based on the independent assessment, resulting in 80 patients in the efficacy population (dextromethorphan-bupropion, N=43; bupropion, N=37). The mean change from baseline in MADRS score over weeks 1-6 (overall treatment effect) was significantly greater with dextromethorphan-bupropion than with bupropion (˜13.7 points vs. −8.8 points; least squares mean difference=−4.9; 95% CI=−3.1, −6.8). MADRS score change with dextromethorphan-bupropion was significantly greater than with bupropion at week 2 and every time point thereafter (week 6: −17.3 vs. −12.1 points; least-squares mean difference=−5.2, 95% CI=−1.1, −9.3). Remission rates were significantly greater with dextromethorphan-bupropion at week 2 and every time point thereafter (week 6:46.5% vs. 16.2%; least-squares mean difference=30.3%, 95% CI=11.2, 49.4). Response rates (≥50% decrease in MADRS score from baseline) at week 6 were 60.5% with dextromethorphan-bupropion and 40.5% with bupropion (least-squares mean difference=19.9%, 95% CI=−1.6, 41). Most secondary outcomes favored dextromethorphan-bupropion. The most common adverse events with dextromethorphan-bupropion were dizziness, nausea, dry mouth, decreased appetite, and anxiety. Dextromethorphan-bupropion was not associated with psychotomimetic effects, weight gain, or sexual dysfunction.

### Conclusions:

In patients with major depression, dextromethorphan-bupropion significantly improved depressive symptoms compared with bupropion and was generally well tolerated.

Major depressive disorder is a prevalent and disabling condition, and it is the leading cause of disability worldwide. Currently approved oral antidepressants work primarily via monoamine pathways. Partial or inadequate response is common with these agents, and they typically take several weeks to produce clinically meaningful effects. In the Sequenced Treatment Alternatives to Relieve Depression (STAR*D) trial, about two-thirds of depression patients failed to achieve remission with first-line treatment, and of those who experienced a clinical response, approximately 60% did so only at or after 8 weeks of treatment.

Involvement of the glutamatergic system in the pathogenesis of depression is suggested by data from neuroimaging, cellular, and clinical studies. Dextromethorphan is an uncompetitive antagonist of the N-methyl-D-aspartate (NMDA) receptor (an ionotropic glutamate receptor) and a sigma-1 receptor agonist. Blockade of the NMDA receptor and agonism of the sigma-1 receptor modulate glutamate signaling in the central nervous system. The clinical utility of dextromethorphan has been limited by its rapid and extensive metabolism through CYP2D6, yielding subtherapeutic plasma levels. A tablet combining dextromethorphan and bupropion (hereafter dextromethorphan-bupropion) has been formulated to increase the bioavailability and half-life of dextromethorphan and has been developed for the treatment of major depression. The bupropion component serves to increase dextromethorphan plasma concentrations by inhibiting its metabolism. A breakthrough therapy designation was granted by the U.S. Food and Drug Administration (FDA) for dextromethorphan-bupropion for the treatment of major depressive disorder in March 2019. This designation is granted to candidate drugs that show potential for benefit above that of available therapies based on preliminary clinical data, and it provides the sponsor with added focus from and greater interactions with FDA staff during the development of the candidate drug.

The high rate of failure to achieve signal detection in depression clinical trials has been well documented. A large analysis of depression clinical trials submitted for new drug applications over a 25-year period revealed a nearly 50% trial failure rate, and a declining treatment effect (drug-placebo difference), over this period. Reasons identified in the literature for the poor signal detection and high failure rate of depression clinical trials include high and rising placebo response rates, site rater biases resulting in baseline score inflation, inclusion of less severely symptomatic patients, and failure to exclude patients who were not eligible for study participation. Approaches to addressing rising placebo rates have included placebo lead-in designs and the sequential parallel comparison design, which remove subjects with high placebo responses to increase the treatment effect. These approaches have met with mixed success. Approaches to reducing the inclusion of ineligible or inappropriate patients include use of a third party to reinterview potential patients to confirm disease severity and eligibility. A limitation of these approaches is that the sites are not blinded to the use of the independent assessment.

In this pilot trial, we utilized measures to potentially address the issues contributing to poor signal detection. These measures included confirmation of disease severity through independent assessment and comprehensive blinding of participating clinical trial sites.

### Methods

### Study Design

This was a 6-week randomized, double-blind, active-controlled phase 2 trial conducted at four sites in the United States from May 2018 to December 2018. The trial used bupropion, an approved antidepressant, as the control because it is a component of dextromethorphan-bupropion.

To address potential site rater biases contributing to inclusion of inappropriate patients, this study evaluated efficacy only in patients whose diagnosis and severity of major depressive disorder were confirmed, based on clinical review, by an independent assessor who was blinded to treatment assignment. The clinical review utilized only documentation collected by the sites at screening and baseline, including a complete medical history and clinician- and patient-reported outcome measures (described below, under Efficacy Assessments). The independent assessor did not have direct contact with study participants or access to any patient data collected after randomization. In order to reduce investigator expectation bias and therefore placebo response, sites were blinded to the primary efficacy variable and the presence of an independent assessor. Sites were provided a blinded protocol that presented the trial as a safety study with exploratory efficacy assessments. Detailed discussion of the efficacy analysis was limited to the statistical analysis plan, which was not provided to the sites.

All sites gained independent review board approval, and all patients provided written informed consent prior to participation. The site investigators gathered the trial data, and Applicant ensured that all persons administering rating scales were qualified and appropriately trained. The trial was conducted in accordance with the International Council on Harmonization's guidelines for Good Clinical Practice, the principles of the Declaration of Helsinki, and all regulatory requirements. The clinical trial was listed on ClinicalTrials.gov (NCT03595579).

### Participants

The study evaluated patients 18-65 years of age with a confirmed diagnosis of major depressive disorder and a current major depressive episode of moderate or greater severity. The diagnosis was established using the DSM-5 criteria for major depressive disorder without psychotic features, based on the Structured Clinical Interview for DSM-5, Clinical Trials Version, and the site investigator's rating of a score ≥25 on the Montgomery-Åsberg Depression Rating Scale (MADRS) and a score ≥4 on the Clinical Global Impressions severity scale (CGI-S). Confirmation of the diagnosis of major depressive disorder and a current major depressive episode of moderate or greater severity was performed by a blinded independent assessor based on a clinical review prior to database lock and study unblinding.

Key exclusion criteria included bipolar disorder, panic disorder, obsessive-compulsive disorder, treatment-resistant depression (defined as having had at least two failed adequate antidepressant treatments in the current major depressive episode), a substance use disorder within the past year, a lifetime history of psychotic disorder, a clinically significant risk of suicide, and a history of seizure disorder. Patients could have been on antidepressant treatment prior to study entry but were required to be completely off the prior treatment, with a washout of at least 1 week or five half-lives of the medication, whichever was longer, prior to the baseline visit and randomization. The screening period was up to 4 weeks to allow for any needed taper and washout of prior medications.

### Randomization and Blinding

Participants were randomized in a 1:1 ratio to receive oral treatment with either dextromethorphan-bupropion or sustained-release bupropion. In order to blind site investigators to the independent confirmation of diagnosis, all patients deemed eligible by the site investigators were randomized to receive study medication and were included in the safety population. As prespecified, patients whose diagnosis or severity was not confirmed by the independent assessor were excluded from the efficacy population.

Dextromethorphan-bupropion tablets and bupropion tablets were identical in appearance. The randomization schedule was computer generated using a permuted block algorithm that randomly allocated study drug to randomization numbers. All patients, investigators, and study personnel were blinded to treatment assignment, and no one involved in the performance of the study had access to the randomization schedule before official unblinding of treatment assignment.

### Procedures

The double-blind treatment period was 6 weeks. Patients received their assigned study medication, dextromethorphan-bupropion (45 mg/105 mg tablet) or bupropion (105 mg tablet), once daily for the first 3 days and twice daily thereafter. The bupropion dosage in the control arm (210 mg daily) was selected to match the dosage incorporated in dextromethorphan-bupropion, enabling an appropriate comparison. During the treatment period, patients tracked their mood daily by completing a daily visual analogue mood scale. Study visits occurred 1, 2, 3, 4, and 6 weeks after the baseline visit. A safety follow-up visit occurred at week 7, 1 week after the last dose of study medication. There were no formal discontinuation criteria; patients were free to withdraw consent for any reason, and investigators were free to remove a patient from the study for any safety-related reason. The dosage of dextromethorphan-bupropion, titrated to twice daily, was selected based on the results of pharmacokinetic trials. Study drug adherence was monitored by counting the number of tablets dispensed and returned and by measurement of plasma concentrations of bupropion at end of study.

### Efficacy Assessments

The primary efficacy variable was the change from baseline to week 6 in the MADRS total score. The MADRS is a 10-item clinician-rated questionnaire ranging from 0 to 60, with higher scores representing more severe depression. The primary hypothesis testing was overall treatment effect on the MADRS score (average of change from baseline for weeks 1-6).

Secondary endpoints included clinical response (defined as a reduction ≥50% from baseline in MADRS total score); remission (defined as a MADRS total score≤10); score on the Clinical Global Impressions improvement scale (CGI-I; scores range from 1 [very much improved] to 7 [very much worse]); score on the CGI-S (scores range from 1 [normal state] to 7 [among the most extremely ill]); score on the 16-item Quick Inventory of Depressive Symptomology-Self-Rated (QIDS-SR; scores range from 0 to 27, with higher scores representing more severe depression); and score on the MADRS-6 (a subscale of the 10-item MADRS evaluating the core symptoms of depression: apparent sadness, reported sadness, inner tension, lassitude, inability to feel, and pessimistic thought).

Safety was assessed based on the incidence of adverse events; changes in vital signs, clinical laboratory measurements, physical examinations, and electrocardiograms; and assessment of suicidal ideation and behavior with the use of the Columbia-Suicide Severity Rating Scale (C-SSRS). Adverse events during the treatment period were defined as adverse events occurring from the time of administration of the first dose of dextromethorphan-bupropion or bupropion until 7 days after the last dose.

### Statistical Analysis

Two patient sets were prespecified for analysis. The safety population included all patients who received at least one dose of study medication. The efficacy population (modified intent-to-treat population) consisted of all patients with a diagnosis of major depressive disorder and a current major depressive episode of moderate or greater severity, confirmed by the independent assessor, who were randomized, received at least one dose of study medication, and had at least one postbaseline assessment.

The primary efficacy variable was the change from baseline in the MADRS total score, and the primary hypothesis testing was overall treatment effect. The overall treatment effect on the MADRS was assessed by averaging the change from baseline at each time point in the study (weeks 1-6). Changes from baseline in MADRS score were analyzed using a mixed model with repeated measures that included treatment (two levels: one indicating active treatment and two indicating the control treatment), week (five levels: weeks 1˜4 and week 6), and treatment-by-week interaction as factors, baseline value as a covariate, and subject as a random effect. This method was used to analyze all other efficacy variables that assessed a change from baseline. Missing values were imputed via the last-observation-carried-forward method.

Overall treatment effects, treatment effects at individual postbaseline weeks, and the differences between treatment effects were estimated using least-squares mean estimation and are reported together with the two-sided 95% confidence interval of the treatment differences. Efficacy variables related to percentages (e.g., clinical response and remission rates) were analyzed via chi-square tests. The CGI-I was analyzed using the Cochran-Mantel-Haenszel test. Analyses were performed using SAS, version 9, and all hypothesis tests were conducted at a two-sided alpha level of 0.05.

If results were found to be positive on the primary hypothesis testing on the MADRS, then other analyses (response, remission) were to be performed on this variable to examine clinical relevance. As this was a phase 2 pilot study, secondary efficacy variables were not adjusted for multiplicity, and nominal p values for these are presented. The sample size assumed approximately 60 patients (30 per arm) with a confirmed diagnosis completing the double-blind period. The sample size of the study was determined based on prior reported experience with trials of a similar stage, in a similar patient population, with a similar objective.

### Results

### Patients and Disposition

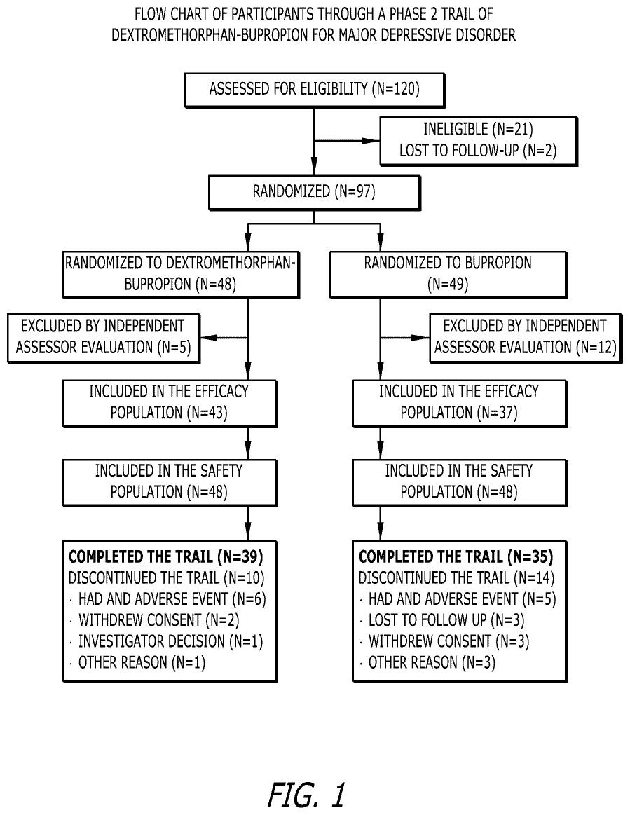

A total of 97 patients were randomized (48 to dextromethorphan-bupropion, 49 to bupropion). Of these, the independent assessor evaluation failed to confirm the diagnosis or severity for 17 patients, resulting in a total of 80 patients in the efficacy (modified intent-to-treat) population (43 in the dextromethorphan-bupropion group, 37 in the bupropion group) (FIG. 1). Of the 17 patients who were excluded from the sample, 16 were excluded because of discrepant depression severity scores (not meeting at least moderate depression severity criteria), and one because the patient did not have a primary diagnosis of major depressive disorder.

FIG. 1 shows the flow chart of participants through a phase 2 trial of Dextromethorphan-bupropion for major depressive disorder.

The patients' baseline demographic and clinical characteristics were generally similar for the dextromethorphan-bupropion and the bupropion groups (Table 1). There was a higher proportion of women, Black or African American patients, and Hispanic or Latino patients in the bupropion group (70.3%, 37.8%, and 29.7%, respectively) compared with the dextromethorphan-bupropion group (58.1%, 27.9%, and 14.0%, respectively). These observed differences were well within random chance (p>0.15) given the small sample sizes. The number of patients in the efficacy population who completed the trial was 34 (79.1%) in the dextromethorphan-bupropion group and 26 (70.3%) in the bupropion group. Completers for the safety population are shown in FIG. 1.

Overall, mean treatment adherence based on tablet counts was high across visits in the safety and efficacy populations (≥90.0% and >91.3%, respectively) for both the dextromethorphan-bupropion and bupropion groups. Consistent with the adherence based on tablet counts, of the 72 patients in the efficacy population with pharmacokinetic samples, 93.1% had measurable study drug concentrations at the last scheduled study visit.

### Efficacy

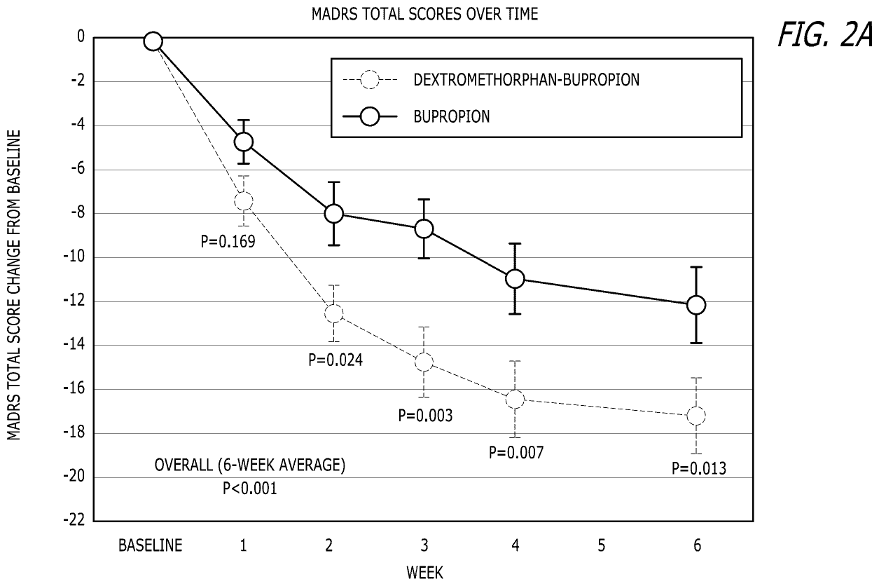

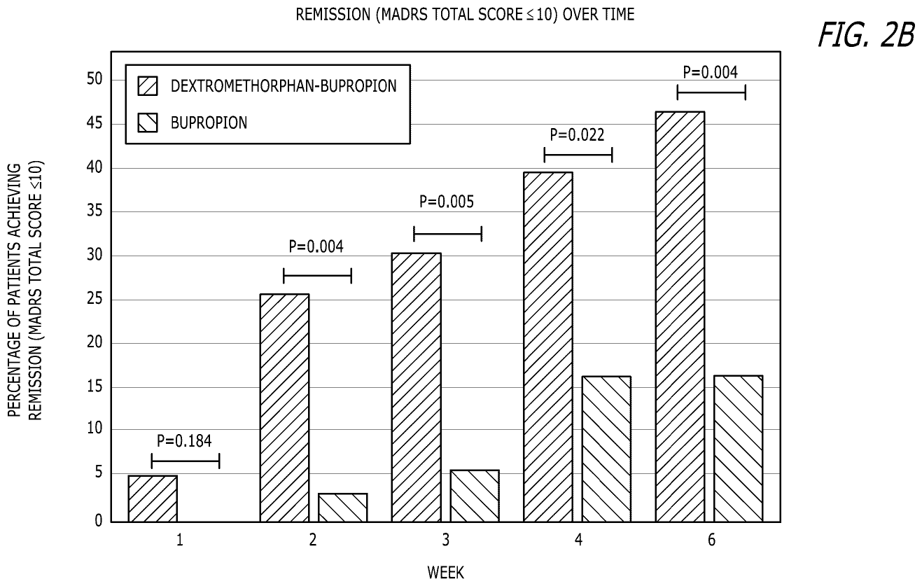

Dextromethorphan-bupropion was significantly associated with reduced MADRS total scores compared with bupropion (FIG. 2). The least-squares mean change from baseline over weeks 1-6 (overall treatment effect) in MADRS total score was −13.7 points in the dextromethorphan-bupropion group and 28.8 points in the bupropion group (least-squares mean difference=−4.9, 95% CI=−3.1, −6.8; Cohen's d=1.2; p<0.001). Improvement with dextromethorphan-bupropion over bupropion on the MADRS total score was observed as early as week 1, with differences being statistically significant starting at week 2 (least-squares mean difference=−4.7, 95% CI=−0.6, −8.8; p=0.024) and at every time point thereafter. At week 6, dextromethorphan-bupropion was associated with a mean change from baseline of −17.3 points in the MADRS total score, compared with −12.1 points for bupropion (least-squares mean difference=−5.2, 95% CI=−1.1, −9.3; Cohen's d=0.6; p=0.013). Results of a sensitivity analysis of the primary endpoint (overall treatment effect) using a mixed model of repeated measures with no imputation of missing values were consistent with the results of the primary analysis (least squares mean difference=−5.1, 95% CI=−3.1, −7.1; p<0.001).

TABLE 1
Baseline demographic and clinical characteristics
of study participants in a phase 2 trial of dextromethorphan-
bupropion for major depressive disorder^{a}

| | Dextromethorphan- | | | |
|---|---|---|---|---|
| Characteristic | Bupropion (N = 43) | | Bupropion (N = 37) | |
| | Mean | SD | Mean | SD |
| Age (years) | 37.3 | 12.0 | 37.7 | 11.9 |
| | N | % | N | % |
| Sex | | | | |
| Female | 25 | 58.1 | 26 | 70.3 |
| Male | 18 | 41.9 | 11 | 29.7 |
| Race | | | | |
| White | 30 | 69.8 | 20 | 54.1 |
| Black or African American | 12 | 27.9 | 14 | 37.8 |
| Asian | 1 | 2.3 | 0 | 0 |
| Other | 0 | 0 | 3 | 8.1 |
| Hispanic or Latino ethnicity | 6 | 14.0 | 11 | 29.7 |
| | Mean | SD | Mean | SD |
| MADRS total score | 31.8 | 4.0 | 32.2 | 4.5 |
| CGI-S score | 4.4 | 0.6 | 4.5 | 0.5 |

^{a}CGI-S = Clinical Global Impressions severity scale; MADRS = Montgomery-Åsberg Depression Rating Scale.

Remission, defined as a MADRS total score≤10, was achieved by a statistically significantly greater percentage of patients in the dextromethorphan-bupropion group than in the bupropion group at week 2 (25.6% and 2.7%, respectively; least-squares mean difference=22.9%, 95% CI=8.8, 36.9; p=0.004) and at every time point thereafter (FIGS. 2A and 2B, Table 2). At week 6, the percentage of patients achieving remission was 46.5% in the dextromethorphan-bupropion group and 16.2% in the bupropion group (least squares mean difference=30.3%, 95% CI=11.2, 49.4; p=0.004). Clinical response, defined as a reduction ≥50% in MADRS total score, favored the dextromethorphan-bupropion group, with the between-group difference reaching statistical significance at week 3 (least-squares mean difference=27.2%, 95% CI=7.2, 47.2; p=0.012) and week 4 (least-squares mean difference=23.0%, 95% CI=1.7, 44.3; p=0.040). At week 6, clinical response was achieved by 60.5% of patients in the dextromethorphan-bupropion, compared with 40.5% in the bupropion group (least-squares mean difference=19.9%, 95% CI=−1.6, 41.5; p=0.075).

Dextromethorphan-bupropion significantly improved MADRS-6 (core symptoms) scores compared with bupropion. The least-squares mean change from baseline over weeks 1-6 (overall treatment effect) on the MADRS-6 score was −10.1 points in the dextromethorphan-bupropion group and −6.6 points in the bupropion group (least-squares mean difference=−3.4, 95% CI=−2.2, −4.7; p<0.001) (Table 2). Improvement with dextromethorphan-bupropion over bupropion on the MADRS-6 score was observed as early as week 1, with differences being statistically significant starting at week 2 (least-squares mean difference=−3.2, 95% CI=−0.4, −6.0; p=0.027) and at every time point thereafter.

Dextromethorphan-bupropion treatment was associated with significantly greater improvement on the CGI-S compared with bupropion. The overall treatment effect (mean improvement from baseline over weeks 1-6) on the CGI-S was −1.6 in the dextromethorphan-bupropion group, compared with −1.0 in the bupropion group (least-squares mean difference=−0.5, 95% CI=−0.3, −0.8; p<0.001) (Table 2). Improvement with dextromethorphan-bupropion over bupropion on the CGI-S was greater compared with bupropion at all time points, with the between-group difference reaching statistical significance at week 2 (least squares mean difference=−0.9, 95% CI=0.0, −1.0; p=0.049) and at every time point thereafter, including week 6 (least-squares mean difference=−0.5, 95% CI=0.0, −1.1; p=0.038). The proportion of patients showing moderate to marked improvement on the CGI-I was statistically significantly greater in the dextromethorphan-bupropion group compared with the bupropion group at week 1 (least-squares mean difference=14.3%, 95% CI=1.5, 28.3; p=0.045) and at week 4 (least-squares mean difference=28.1%, 95% CI=4.1, 52.1; p=0.025).

The dextromethorphan-bupropion group also showed a statistically significant improvement on the patient-rated QIDS-SR compared with the bupropion group. The least squares mean change from baseline over weeks 1-6 (overall treatment effect) on the QIDS-SR was −6.0 for the dextromethorphan-bupropion group and −4.8 for the bupropion group (least-squares mean difference=−1.2, 95% CI=−0.2, −2.2; p=0.017). Remission on the QIDS-SR (defined as a score≤5) at week 6 was achieved by a statistically significantly greater proportion of the dextromethorphan-bupropion group (46.5%) compared with the bupropion group (21.6%) (least-squares mean difference=24.9%, 95% CI=4.9, 44.8; p=0.020).

FIGS. 2A and 2B show the MADRS total scores and remission over time in a phase 2 trial of dextromethorphan-bupropion for major depressive disorder. FIG. 2A displays the mean change from baseline in the MADRS total score over time; FIG. 2B displays the percentage of patients achieving remission (a MADRS total score≤10) over time. MADRS represents Montgomery-Åsberg Depression Rating Scale. Error bars indicate standard error, and p values are calculated via chi-square tests.

TABLE 2
Primary and secondary outcomes in a phase 2 trial of dextromethorphan-
bupropion for major depressive disorder^{a}

| | LS mean | SE | LS mean | SE | LS mean | 95% CI | Cohen's d | p |
|---|---|---|---|---|---|---|---|---|
| MADRS total score, | | | | | | | | |
| change from baseline | | | | | | | | |
| Overall (primary endpoint) | −13.7 | 0.6 | −8.8 | 0.7 | −4.9 | −3.1, −6.8 | 1.2 | <0.001 |
| Week 2 | −12.5 | 1.4 | −7.8 | 1.5 | −4.7 | −0.6, −8.8 | 0.5 | 0.024 |
| Week 6 | −17.3 | 1.4 | −12.1 | 1.5 | −5.2 | −1.1, −9.3 | 0.6 | 0.013 |

| | N | % | N | % | % | 95% CI | NNT | p |
|---|---|---|---|---|---|---|---|---|
| Remission (MADRS score ≤10) | | | | | | | | |
| Week 2 | 11 | 25.6 | 1 | 2.7 | 22.9 | 8.8, 36.9 | 5 | 0.004 |
| Week 6 | 20 | 46.5 | 6 | 16.2 | 30.3 | 11.2, 49.4 | 4 | 0.004 |
| Clinical response (≥50% | | | | | | | | |
| decrease in MADRS score | | | | | | | | |
| from baseline) | | | | | | | | |
| Week 2 | 16 | 37.2 | 10 | 27.0 | 10.2 | −10.2, 30.5 | 10 | ns |
| Week 6 | 26 | 60.5 | 15 | 40.5 | 19.9 | −1.6, 41.5 | 5 | ns |

| | LS mean | SE | LS mean | SE | LS mean | 95% CI | Cohen's d | p |
|---|---|---|---|---|---|---|---|---|
| MADRS-6 score, | | | | | | | | |
| change from baseline | | | | | | | | |
| Overall | −10.1 | 0.4 | −6.6 | 0.5 | −3.4 | −2.2, −4.7 | 1.2 | <0.001 |
| Week 2 | −91 | 10 | −6.0 | 1.0 | −3.2 | −0.4, −6.0 | 0.5 | 0.027 |
| Week 6 | −12.6 | 10 | −8.7 | 1.0 | −3.9 | −1.1, −6.7 | 0.6 | 0.007 |
| CGI-S score, | | | | | | | | |
| change from baseline | | | | | | | | |
| Overall | −1.6 | 0.1 | −1.0 | 0.1 | −0.5 | −0.3, −0.8 | 1.0 | <0.001 |
| Week 2 | −1.4 | 0.2 | −0.9 | 0.2 | −0.5 | −0.0, −1.0 | 0.4 | 0.049 |
| Week 6 | −2.1 | 0.2 | −1.5 | 0.2 | −0.5 | −0.0, −1.1 | 0.5 | 0.038 |

^{a}CGI-S5 Clinical Global Impressions severity scale; LS = least-squares; MADRS = Montgomery-Åsberg Depression Rating Scale; MADRS-6 = 6-item Montgomery Åsberg Depression Rating Scale; NNT = number needed to treat; ns = not significant. Changes from baseline variables were analyzed using a mixed model for repeated measures. This analysis of covariance mixed-effect model for repeated measures included treatment, week, and treatment-by-week interaction as factors, baseline value as a covariate, and patient as a random effect. Treatment effects and treatment differences at each time point were estimated using the least-squares mean estimates. Efficacy variables related to percentages were analyzed via chi-square tests. All hypothesis tests were conducted at a two-sided alpha level of 0.05.

### Safety

The percentage of patients who experienced any adverse events during the treatment period was 72.9% in the dextromethorphan-bupropion group and 64.6% in the bupropion group. The most common adverse events in the dextromethorphan-bupropion group were dizziness, nausea, dry mouth, decreased appetite, and anxiety. In the bupropion group, the most frequently reported adverse events were nausea, headache, dry mouth, decreased appetite, and constipation. The majority of adverse events were mild or moderate in intensity. Adverse events rated of severe intensity were reported by three patients in the dextromethorphan-bupropion group (dizziness in two patients; somnolence, nausea, and anxiety in one patient each) and by one patient in the bupropion group (psychotic disorder). There were no serious adverse events (including hospitalizations) in the trial. The incidence of adverse events resulting in discontinuation of study medication was identical in the dextromethorphan-bupropion and bupropion groups (12.5%). Dizziness and anxiety led to withdrawal of study drug in two patients each in the dextromethorphan-bupropion group, and psychotic disorder in one patient in the bupropion group. Dextromethorphan-bupropion was not associated with psychotomimetic effects, weight gain, or increased sexual dysfunction (Table 3).

Mean changes from baseline in clinical laboratory parameters and vital signs were small and similar between treatment groups. Two patients in the dextromethorphan-bupropion group had adverse events of decreased weight, and one patient in the bupropion group had an adverse event of increased blood pressure, all of which resolved. The adverse events of decreased weight were not serious and were of mild severity.

There were no signals for increased suicidality in this study, as assessed by changes from baseline on the C-SSRS. At baseline, five patients (10.4%) in the dextromethorphan-bupropion group and four patients (8.2%) in the bupropion group gave a positive response on item 1 of the C-SSRS (wish to be dead). At week 1, only two patients in the dextromethorphan-bupropion group gave a positive response to item 1, compared with five patients in the bupropion group. At week 6, one patient in each treatment group gave a positive response on item 1 of the C-SSRS.

TABLE 3
Summary of adverse events in a phase 2 trial of dextromethorphan-
bupropion for major depressive disorder^{a}

| | Dextromethorphan- | | | |
|---|---|---|---|---|
| Event | Bupropion (N = 48) | | Bupropion (N = 48) | |
| | N | % | N | % |
| Any adverse event | 35 | 72.9 | 31 | 64.6 |
| Serious adverse event^{b} | 0 | 0.0 | 0 | 0.0 |
| Severe adverse event^{c} | 3 | 6.3 | 1 | 2.1 |
| Adverse event leading to discontinuation | 6 | 12.5 | 6 | 12.5 |
| of study drug | | | | |
| Adverse events occurring in ≥5 patients | | | | |
| in either treatment group | | | | |
| Dizziness | 10 | 20.8 | 2 | 4.2 |
| Nausea | 8 | 16.7 | 6 | 12.5 |
| Dry mouth | 5 | 10.4 | 4 | 8.3 |
| Decreased appetite | 5 | 10.4 | 4 | 8.3 |
| Anxiety | 5 | 10.4 | 1 | 2.1 |
| | Mean | SD | Mean | SD |
| Change from baseline in body weight at | −0.6 | 2.4 | 0.1 | 2.1 |
| week 6 (kg) | | | | |

^{a}The safety population included all randomized patients who received at least one dose of dextromethorphan-bupropion or bupropion. Adverse events that occurred during the treatment period were defined as those that started or worsened from the time of the first dose of dextromethorphan-bupropion or bupropion through 7 days after the last dose.
^{b}A serious adverse event was defined as any adverse event that resulted in death, was immediately life threatening, led to inpatient hospitalization or prolongation of hospitalization, or caused persistent or clinically significant disability or incapacity.
^{c}A severe adverse event was defined as any event that interrupted usual activities of daily living, or significantly affected clinical status, or required intensive therapeutic intervention.

### Discussion

In this randomized controlled trial, dextromethorphan-bupropion demonstrated rapid, substantial, and statistically significant antidepressant efficacy compared with the active comparator bupropion on the primary efficacy variable (MADRS total score) and multiple other clinician and patient-reported measures of depression severity. Given the known challenges in achieving signal detection in depression clinical trials, design features were implemented in this study to increase assay sensitivity and ensure evaluation of the appropriate patient population. To address potential site rater biases toward the inclusion of inappropriate patients, this study evaluated only patients whose diagnosis and severity of major depressive disorder were confirmed by a blinded independent assessor, based on clinical review. Another important design feature implemented to increase the ability to detect an efficacy signal in this phase 2 trial was the blinding of site investigators to the efficacy objective of the trial to reduce expectation bias.

Dextromethorphan-bupropion rapidly reduced depressive symptoms, as measured by the MADRS total score, as early as week 1, with statistically significant differences over bupropion observed by week 2 and at every time point thereafter. The treatment difference on the MADRS (dextromethorphan-bupropion change minus bupropion change) was substantial and consistent over time, being approximately 5 points at each time point from week 2 through week 6 (range, 4.5-5.6 points). This treatment effect over an active comparator compares favorably to the approximately 2.5-point mean difference from placebo seen at 6-8 weeks in antidepressant studies in the FDA database.

Symptom remission is considered a desired goal in depression treatment because it is associated with better daily functioning and better long-term prognosis. Rates of remission on the MADRS (a total score≤10) were statistically significantly greater in the dextromethorphan-bupropion group starting at week 2 (p=0.004) and at every time point thereafter. Rates of remission on the patient-rated QIDS-SR (a total score≤5) at week 6 similarly favored dextromethorphan-bupropion (p=0.020), demonstrating consistency between clinician- and patient-reported outcomes.

The MADRS core symptom subscale (MADRS-6) was evaluated because it has been suggested that core symptoms may be more sensitive to change from antidepressant treatment. Results on the MADRS-6 were consistent with those on the 10-item MADRS, demonstrating greater improvements among patients in the dextromethorphan-bupropion group compared with those in the bupropion group, with statistical significance achieved on both measures starting at week 2 and at every time point thereafter.

Results of clinicians' global assessments were consistent with the results from the symptom-specific scales and favored dextromethorphan-bupropion. Clinicians reported rapid and consistently greater reduction in disease severity, measured by the CGI-S, with dextromethorphan-bupropion compared with bupropion, which was statistically significant starting at week 2 (p=0.049) and at every time point thereafter, including week 6 (p=0.038).

Dextromethorphan-bupropion demonstrated a rapid onset of effect on depressive symptoms and global measures, with statistically significant improvements compared with bupropion observed starting at week 2 on several measures, despite the small sample size of the study. Dextromethorphan-bupropion demonstrated a statistically significant reduction in MADRS total score from baseline compared with bupropion starting at week 2 (p=0.024). Remission rates on the MADRS were statistically significantly superior in the dextromethorphan-bupropion group compared with the bupropion group starting at week 2 (26% compared with 3%; p=0.004). Global measures showed rapid improvement with dextromethorphan-bupropion, with statistically significant changes observed at week 1 on the CGI-I (p=0.045) and at week 2 on the CGI-S (p=0.049).

This study is the first controlled trial of dextromethorphan-bupropion in patients with depression. Murrough et al. reported results of the combination of dextromethorphan and quinidine in patients with treatment-resistant depression. However, that was an open-label trial with no control arm, which limits interpretation. The development of the combination of deuterated dextromethorphan and quinidine for the treatment of major depression was halted because results of a phase 2 placebo-controlled trial in this indication (NCT02153502) did not provide sufficient evidence of efficacy to justify continued development. In the present phase 2 trial, the bupropion arm (210 mg/day) performed as expected, resulting in a substantial reduction (12 points) from baseline in MADRS total score at week 6. By comparison, in an 8-week, 362-patient, randomized, placebo-controlled trial, bupropion treatment at dosages of 150 mg/day or 300 mg/day resulted in a reduction of approximately 10 points from baseline in Hamilton Depression Rating Scale (HAM-D) score for each of the two dosages at week 8. This 10-point HAM-D change translates to an approximately 12-point change on the MADRS, which is in line with the bupropion result in the present phase 2 trial. The bupropion dosage in the present trial was selected to match the dosage incorporated in dextromethorphan-bupropion, enabling an appropriate comparison. This dosage is lower than the usual target dosage of 300 mg/day as monotherapy stated in the FDA prescribing information for bupropion.

Dextromethorphan-bupropion was safe and well tolerated in this trial. The incidences of adverse events were generally similar between the two groups except for dizziness, which was reported in 20.8% of the dextromethorphan-bupropion group, compared with 4.2% of the bupropion group. To estimate the true incidence of dizziness with dextromethorphan-bupropion, larger studies have been performed (ClinicalTrials.gov identifier: NCT04019704). Rates of discontinuations due to adverse events were similar in the two treatment arms.

Unlike other NMDA receptor antagonists, dextromethorphan-bupropion was not associated with psychotomimetic effects. This tolerability profile could be related to the significantly faster rate of unblocking of the NMDA receptor channel reported for dextromethorphan as compared to other NMDA antagonists. Dextromethorphan-bupropion was not associated with abuse-related adverse events in this trial. While dosing in this trial was only 6 weeks in duration, dextromethorphan-bupropion has been dosed for up to 1 year in a long-term open-label safety trial (ClinicalTrials.gov identifier: NCT04039022). In that and other, larger controlled trials, dextromethorphan-bupropion to date has not been associated with abuse. Dextromethorphan-bupropion was not associated with weight gain or increased sexual dysfunction in this 6-week trial.

Limitations of this trial include exclusion of patients with inadequate symptom severity, psychotic or other psychiatric disorders, substance use disorders, clinically significant risk of suicide, or significant medical comorbidities. These exclusions, along with prohibition of certain concomitant medications, may limit the generalizability of the study findings. In addition, treatment at experienced trial sites by specialized clinicians under a trial protocol with frequent clinical assessments may not reflect general practice. While most of the secondary outcomes favored dextromethorphan-bupropion over bupropion, the confidence intervals for the between-group differences were not adjusted for multiple comparisons. A greater number of patients in the bupropion arm were excluded by the independent assessment compared with the dextromethorphan-bupropion arm, which may affect the overall results. The imputation of missing values via the last-observation-carried-forward method may affect the results of categorical variables such as response and remission. Finally, the trial duration was limited to 6 weeks. The effect of long-term treatment with dextromethorphan-bupropion has been evaluated in a separate study. In this phase 2 randomized clinical trial, treatment with dextromethorphan-bupropion resulted in clinically meaningful and statistically significant improvements in depressive symptoms compared with the active comparator bupropion, and was well tolerated. Reductions in depressive symptoms with dextromethorphan-bupropion were statistically significant at week 2 and at every time point thereafter compared with bupropion alone.

### Example 2

### Dextromethorphan-Bupropion Achieves Primary and Key Secondary Endpoints in the MERIT Phase 2 Trial in Treatment Resistant Depression

Dextromethorphan-Bupropion significantly delayed the time to relapse of depression compared to placebo (p=0.002, primary endpoint).

Dextromethorphan-Bupropion significantly prevented relapse of depression over at least 6 months compared to placebo (p=0.004, key secondary endpoint).

Dextromethorphan-Bupropion, a novel, oral, investigational NMDA receptor antagonist with multimodal activity, met the primary and key secondary endpoints in the MERIT (Mechanistic Evaluation of Response in TRD) Phase 2 trial, and substantially and statistically significantly prevented relapse of depressive symptoms compared to placebo in patients with treatment resistant depression (TRD). The MERIT study was a randomized, double-blind, placebo-controlled, relapse prevention, multi-center, U.S. trial, which-evaluated 44 TRD patients. Patients in stable remission after treatment with Dextromethorphan-Bupropion were randomized to continue treatment with Dextromethorphan-Bupropion or to discontinue Dextromethorphan-Bupropion and switch to placebo.

Dextromethorphan-Bupropion met the primary endpoint by substantially and statistically significantly delaying the time to relapse of depressive symptoms as compared to placebo (p=0.002), with no relapses observed with Dextromethorphan-Bupropion over at least 6 months of double-blind treatment. Dextromethorphan-Bupropion also met the key secondary endpoint of relapse prevention, based on the rates of relapse during the double-blind treatment period (0.0% of Dextromethorphan-Bupropion patients, 36.4% of patients switched from Dextromethorphan-Bupropion to placebo, p=0.004).

Dextromethorphan-Bupropion was well tolerated in the trial. There were no treatment-emergent adverse events reported in >1 patient in the Dextromethorphan-Bupropion group. One subject in the Dextromethorphan-Bupropion group experienced two serious adverse events (gout and bacteremia), both of which were deemed not related to the study medication.

### About the MERIT Study

MERIT was a Phase 2, randomized, double-blind, placebo-controlled, multi-center study to evaluate Dextromethorphan-Bupropion compared to placebo in preventing relapse of depressive symptoms in patients with treatment resistant depression (TRD). Treatment resistance was defined as ongoing symptoms of depression despite receiving treatment with two or more prior antidepressants during the current major depressive episode. TRD patients were enrolled into MERIT from the long-term, open-label Phase 3 trial of Dextromethorphan-Bupropion, and were required to be in stable remission prior to randomization. Stable remission was defined as at least two consecutive Montgomery-Åsberg Depression Rating Scale (MADRS) scores of ≤12, separated by at least 4 weeks.

A total of 44 TRD patients who experienced a stable remission after up to 12 months of open-label treatment with Dextromethorphan-Bupropion (45 mg dextromethorphan-105 mg bupropion) tablets twice daily, were randomized 1:1 to continue Dextromethorphan-Bupropion, or to discontinue Dextromethorphan-Bupropion and switch to placebo, in a double-blind fashion, for at least 26 weeks or until a relapse of depressive symptoms occurred. Relapse was defined in the study by one or more of the following: MADRS total score ≥18 for 2 consecutive assessments; a ≥2-point increase from randomization in the Clinical Global Impression of Severity, with a minimum CGI-S score of 4, for 2 consecutive assessments; hospitalization due to worsening of depression or risk of suicide; investigator determination of relapse or need for additional antidepressant or treatment switch.

The primary endpoint in the study was time from randomization to relapse calculated by the Kaplan-Meier estimates and the hazard ratio. The key secondary endpoint, to assess relapse prevention, was the percentage of patients without relapse.

### About Major Depressive Disorder

Major depressive disorder (MDD) is a debilitating, chronic, biologically-based disorder characterized by low mood, inability to feel pleasure, feelings of guilt and worthlessness, low energy, and other emotional and physical symptoms, and which impairs social, occupational, educational, or other important functioning. In severe cases, MDD can result in suicide. According to the National Institutes of Health, an estimated 7% of U.S. adults, or approximately 19 million, experience MDD each year. According to the World Health Organization (WHO), depression is the leading cause of disability worldwide, and is a major contributor to the overall global burden of disease. Nearly two-thirds of diagnosed and treated patients do not experience adequate treatment response with currently available first-line therapy, highlighting the need for additional therapies with new mechanisms of action. The majority of initial failures also fail second-line treatment. Patients diagnosed with MDD are defined as having treatment resistant depression (TRD) if they have failed to respond to two or more antidepressant therapies.

### About Dextromethorphan-Bupropion

Dextromethorphan-bupropion is a novel, oral, patent-protected, investigational NMDA receptor antagonist with multimodal activity under development for the treatment of major depressive disorder and other central nervous system (CNS) disorders. Dextromethorphan-Bupropion utilizes a proprietary formulation and dose of dextromethorphan and bupropion, and Applicant's metabolic inhibition technology, to modulate the delivery of the components. The dextromethorphan component of Dextromethorphan-Bupropion is an uncompetitive N-methyl-D-aspartate (NMDA) receptor antagonist, also known as a glutamate receptor modulator, which is a novel mechanism of action, meaning it works differently than currently approved oral therapies for major depressive disorder. The dextromethorphan component of Dextromethorphan-Bupropion is also a sigma-1 receptor agonist. The bupropion component of Dextromethorphan-Bupropion serves to increase the bioavailability of dextromethorphan, and is a norepinephrine and dopamine reuptake inhibitor. Dextromethorphan-Bupropion has been granted U.S. Food and Drug Administration (FDA) Breakthrough Therapy designations for the treatment of MDD and for treatment of Alzheimer's disease agitation.

### Example 3

### Efficacy and Safety of Dextromethorphan-Bupropion, an Oral NMDA Receptor Antagonist, in the Prevention of Relapse in Patients With Treatment-Resistant Depression:

Results from MERIT, a Double-Blind, Placebo-Controlled Relapse Prevention Trial

### Introduction

Major depressive disorder (MDD) is a serious disorder: MDD is a chronic, disabling, prevalent, biologically-based disorder, and a leading cause of suicide.

Treatment-Resistant Depression (TRD): About 40% of patients with MDD are treatment resistant, defined as non-response to two or more antidepressants correctly dosed from 2 different classes within the current depressive episode.

Relapse of MDD is not uncommon in patients with TRD who eventually achieve remission, with >40% of patients relapsing within ˜4 months of treatment.

Need for mechanistically novel approaches: Currently approved oral antidepressants work primarily through monoaminergic mechanisms.

Glutamatergic hypothesis of MDD: Clinical and preclinical evidence has implicated dysfunctional glutamatergic neurotransmission in the pathophysiology of MDD, suggesting a role for NMDA receptor antagonism in the treatment of MDD.

There is an urgent clinical need for: New, more effective, faster-acting, mechanistically novel, and well-tolerated MDD treatments.

Dextromethorphan-Bupropion is a Novel, Oral, Investigational NMDA Receptor Antagonist with Multimodal Activity:

The dextromethorphan component of Dextromethorphan-Bupropion is an antagonist of the NMDA receptor, an ionotropic glutamate receptor, and a sigma-1 receptor agonist.

These actions modulate glutamatergic neurotransmission.

The bupropion component of Dextromethorphan-Bupropion serves primarily to increase the bioavailability of dextromethorphan, and is a norepinephrine and dopamine reuptake inhibitor.

### MERIT Trial Objective

The MERIT (Mechanistic Evaluation of Response in TRD) was a Phase 2, randomized, double-blind, placebo-controlled, relapse prevention study designed to evaluate Dextromethorphan-Bupropion compared to placebo in preventing relapse of depressive symptoms in patients with TRD.

This trial is registered on ClinicalTrials.gov (NCT04608396).

### Study Procedures

TRD patients in stable remission after treatment with Dextromethorphan-Bupropion (N=44) were randomized to continue treatment with Dextromethorphan-Bupropion or to discontinue Dextromethorphan-Bupropion and switch to placebo for up to 52 weeks, or until relapse.

TRD patients who achieved stable remission in the open-label, long-term study of Dextromethorphan-Bupropion (‘COMET’) were eligible to be enrolled into MERIT.

Stable remission was defined as at least two consecutive Montgomery-Åsberg Depression Rating Scale (MADRS) scores of ≤12, separated by at least 4 weeks.

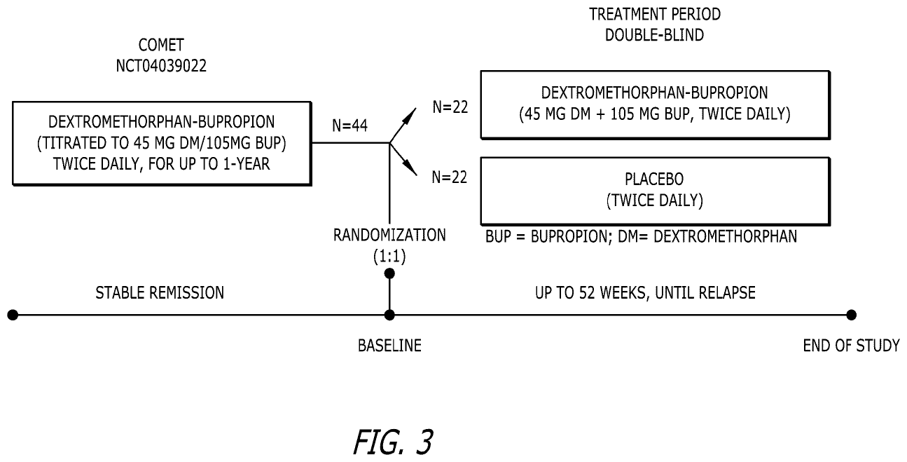

FIG. 3 shows the study procedure.

### Eligibility Criteria

### Key Inclusion Criteria:

Stable responder, defined as at least two consecutive MADRS scores of ≤12, separated by at least 4 weeks.

Subject's depression was treatment resistant prior to entering COMET, defined as:
Prior enrollment in a study evaluating Dextromethorphan-Bupropion in patients with prospectively defined TRD (NCT02741791), or
Subjects enrolled in COMET who had received 2 or more prior antidepressants during their major depressive episode prior to entering COMET.

### Key Exclusion Criteria:

History of electroconvulsive therapy, vagus nerve stimulation, transcranial magnetic stimulation, or any experimental central nervous system treatment during the current episode or in the past 6 months;

Schizophrenia, bipolar disorder, obsessive compulsive disorder; and

Psychiatric symptoms secondary to any other general medical condition.

### Efficacy Assessments

Primary Endpoint: Time from Randomization to Relapse Calculated by the Kaplan-Meier Estimates

Subjects withdrawing for reasons other than relapse were considered not to have relapsed and were censored.

Key Secondary Endpoint: Percentage of Subjects without Relapse

Relapse was defined by ≥1 of the following:
MADRS total score ≥18 for 2 consecutive assessments, separated by 7 to 21 days;
≥2-point increase from randomization on the Clinical Global Impression of Severity (CGI-S), with a minimum CGI-S score of 4, for 2 consecutive assessments, separated by 7 to 21 days;
Loss of therapeutic response, such as:
   (i) Hospitalization due to worsening of depression or due to risk of suicide.
   (ii) Investigator-determined need for additional antidepressant treatment or treatment switch.
   (iii) Patient had relapsed in the opinion of the investigator.

### Results

### Demographics and Baseline Characteristics

Table 4 shows the demographics and baseline characteristics.

TABLE 4

| | DM-BUP | | Placebo | |
|---|---|---|---|---|
| Characteristics | (N = 22) | | (N = 22) | |
| Age, years | 49.9 | (11.1) | 51.1 | (13 .4) |
| Sex, n (%) | | | | |
| Male | 9 | (40.9%) | 7 | (31.8%) |
| Female | 13 | (59.0%) | 15 | (68.2%) |
| Race, n (%) | | | | |
| White | 17 | (77.3%) | 18 | (81.8%) |
| Black or African American | 5 | (22.7%) | 4 | (18.2%) |
| BMI (kg/m²) | 31.6 | (6.72) | 29.4 | (5.92) |
| MADRS Total Score at randomization | 4.0 | (3.29) | 5.9 | (3.89) |
| CGI-S Score at Randomization | 1.3 | (0.79) | 1.6 | (0.79) |

CGI-S = Clinical Global Impression-Severity; Values are mean (SD) unless otherwise noted.
DM = Dextromethorphan; BUP = Bupropion.

A total of 22 subjects were randomized to each treatment group.

Mean age in both treatment groups was approximately 50 years.

The majority of subjects were white and female.

Subjects were in remission, with mean MADRS scores of <6 and CGI-S scores <2.

### Primary Endpoint: Time From Randomization to Relapse

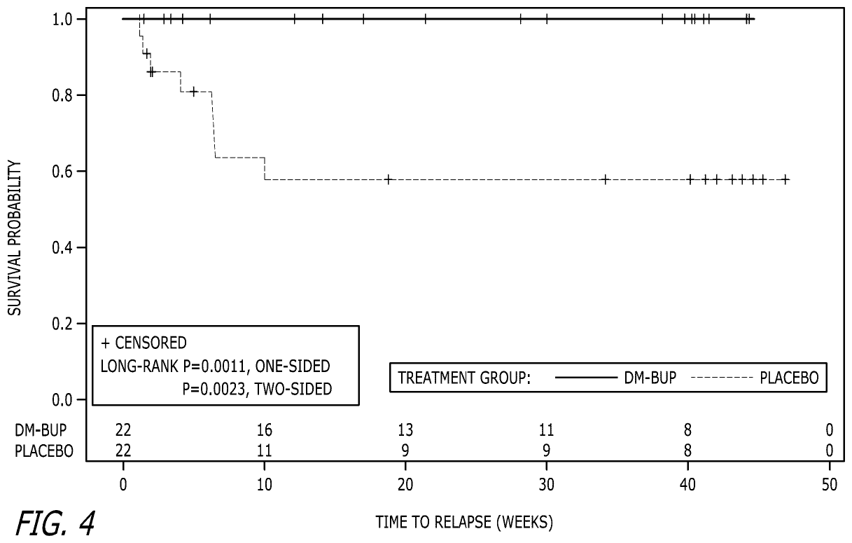

FIG. 4 shows the time to relapse in weeks.

As shown in FIG. 4, the following were observed.

Dextromethorphan-Bupropion achieved the primary endpoint by delaying time to relapse of depressive symptoms.

Subjects continuing treatment with Dextromethorphan-Bupropion demonstrated a substantially and statistically delayed time to relapse of depressive symptoms compared to those randomized to placebo (p=0.0023).

Key Secondary Endpoint: Percent of Patients without Relapse

The percents of patients without relapse for Dextromethorphan-Bupropion group and placebo group are shown in Table 5.

TABLE 5

| | | | Difference (%) | |
|---|---|---|---|---|
| | DM-BUP | Placebo | DM-BUP vs Placebo | |
| Result | (N = 22) | (N = 22) | (95% CI) | P-value |
| Relapse | 0 (0.0) | 8 (36.4) | 36.4 (16.3, 56.5) | 0.004 |
| Without relapse | 22 (100.0) | 14 (63.6) | | |

CI = confidence interval.
Values are n (%) unless otherwise noted.

Treatment with Dextromethorphan-Bupropion prevented relapse in a statistically significant greater proportion of subjects as compared to placebo.

No subjects treated with Dextromethorphan-Bupropion relapsed over the evaluation period compared to 8 subjects (36.4%) receiving placebo, for a relapse rate difference of 36.4% (p=0.004)

### Percentage of Subjects Meeting Each Relapse Criterion

The relapse criteria is shown in Table 6.

TABLE 6

| | DM-BUP | Placebo | |
|---|---|---|---|
| Relapse Criteria | (N = 22) | (N = 22) | |
| Any relapse criteria met | 0 (0.0) | 8 | (36.4) |
| MADRS ≥18 for 2 consecutive assessments 7- | 0 (0.0) | 6 | (27.3) |
| 21 days apart | | | |
| ≥2-point increase from randomization in | 0 (0.0) | 1 | (2.4) |
| CGI-S, with a minimum score of 4, for 2 | | | |
| consecutive assessments | | | |
| Hospitalization due to worsening of depression | 0 (0.0) | 0 | (0.0) |
| or due to risk of suicide | | | |
| Investigator determined need for additional | 0 (0.0) | 0 | (0.0) |
| antidepressant treatment or treatment switch | | | |
| Patient had relapsed in opinion of Investigator | 0 (0.0) | 1 | (4.5) |

The most common reason for relapse in the placebo group were MADRS of ≥18 for two consecutive visits.

No subjects in the Dextromethorphan-Bupropion group met the MADRS relapse criterion, or any of the other final criteria.

Seven subjects in the Dextromethorphan-Bupropion group had a 2-point increase in the CGI-S but did not meet relapse criteria as their scores did not exceed 3 (“mildly depressed”), and/or the excursions were transient (not sustained for two consecutive assessments).

### Safety and Tolerability

Table 7 shows the treatment-emergent adverse events (TEAE).

TABLE 7

| | DM-BUP | Placebo | |
|---|---|---|---|
| Treatment-Emergent Adverse Event (TEAE) | (N = 22) | (N = 22) | |
| Headache | 1 (4.5%) | 3 | (13.6%) |
| Urinary tract infection | 1 (4.5%) | 1 | (4.5%) |
| Road traffic accident | 1 (4.5%) | 1 | (4.5%) |
| Abdominal pain | 1 (4.5%) | 0 | (0%} |
| Anxiety | 1 (4.5%) | 0 | (0%) |
| Gout | 1 (4.5%) | 0 | (0%) |
| Insomnia | 1 (4.5%) | 0 | (0%) |
| Ligament sprain | 1 (4.5%) | 0 | (0%) |
| Middle insomnia | 1 (4.5%) | 0 | (0%) |
| Seasonal allergy | 1 (4.5%) | 0 | (0%) |
| Somnolence | 1 (4.5%) | 0 | (0%) |
| Staphylococcus test positive | 1 (4.5%) | 0 | (0%) |

Long-term treatment with Dextromethorphan-Bupropion was well tolerated.

One subject in the Dextromethorphan-Bupropion group experienced two serious adverse events (gout, Staphylococcus test positive), both of which were deemed not related to study drug, and one subject in the Dextromethorphan-Bupropion group had a TEAE that led to study drug withdrawal (anxiety).

### Conclusions

Dextromethorphan-Bupropion substantially and statistically significantly delayed the time to relapse of depressive symptoms compared to placebo in TRD patients that had achieved stable remission on Dextromethorphan-Bupropion.

No relapses were observed in patients continuing Dextromethorphan-Bupropion over at least 6 months of double-blind treatment.

Dextromethorphan-Bupropion was well tolerated in the trial and no new safety signals were observed.

These data support the continued development of Dextromethorphan-Bupropion in TRD and provide additional evidence of the long-term effectiveness of Dextromethorphan-Bupropion in the treatment of MDD.

### Example 4

Dextromethorphan-Bupropion Improves Depressive Symptoms and Functioning in Patients with One Prior Treatment Failure: Results from the EVOLVE Long-Term, Open-Label Study

### Introduction

Major depressive disorder (MDD) is a serious disorder: MDD is a chronic, disabling, prevalent, biologically-based disorder, and a leading cause of suicide.

MDD is difficult to treat: In the largest open-label study conducted, STAR*D, only about ⅓ of individuals with MDD achieved remission with up to 12 weeks of therapy with the SSRI citalopram.

Second line treatment: In STAR*D, following non-remission with an SSRI, remission rates for second line treatments were about 25% regardless of the switch strategy employed: switching to a different SSRI (sertraline), switching to a SNRI (venlafaxine), or switching to a NDRI (bupropion).

Need for mechanistically novel approaches: The declining remission rates in STAR*D may be partially explained by the lack of pharmacological diversity amongst the different treatments, e.g., all antidepressants employed are thought to work in generally the same way: monoamine modulation.

Glutamatergic hypothesis of MDD: Clinical and preclinical evidence has implicated dysfunctional glutamatergic neurotransmission in the pathophysiology of MDD, suggesting a role for NMDA receptor antagonism in the treatment of MDD.

There is an urgent clinical need for: new, more effective, faster-acting, mechanistically novel, and well-tolerated MDD treatments.

### Objective

The objective is to evaluate the safety and efficacy of Dextromethorphan-Bupropion in MDD patients who had been treated with one prior treatment in their current major depressive episode.

### Study Design: EVOLVE

EVOLVE (Evaluation of NMDA Modulation for Depressive Episodes) was an open-label, US trial, in which patients were treated with Dextromethorphan-Bupropion (45 mg dextromethorphan HBr-105 mg bupropion HCl) twice daily for up to 15 months.

Eligible patients had either rolled in following completion of a prior Dextromethorphan-Bupropion study (MERIT), or were directly enrolled, and had a DSM-5 diagnosis of MDD, a MADRS score of ≥25, and had been treated with at least 1 prior antidepressant in the current major depressive episode.

A total of 186 patients were enrolled, consisting of 35 roll-over and 146 directly enrolled. Here we present the results for the directly enrolled patients.

Newly enrolled subjects whose MADRS score did not decrease by ≥25% at Week 6 were discontinued from the study.

### Efficacy Outcome Measures:

The primary efficacy analyses were the change from baseline to Week 6 (primary timepoint) and Weeks 1 and 2 (key secondary timepoints):
1) Montgomery-Åsberg Depression Rating Scale (MADRS):
   Clinical Response (≥50% reduction in MADRS total score);
   Clinical Remission (≤10 on the MADRS total score).
2) Sheehan Disability Scale (SDS):
   Clinical Response in Functioning (≤12 on the SDS total score);
   SDS Remission (≤6 total score).

### Statistical Analysis:

Efficacy analyses were conducted on the mITT population which consisted of all patients who received at least 1 dose of Dextromethorphan-Bupropion and provided at least 1 post-baseline efficacy measurement.

Safety analysis were conducted on the safety population, which included all patients who received at least 1 dose of Dextromethorphan-Bupropion.

Change from baseline P-values were analyzed using paired t-test.

Table 8 shows the Key Inclusion and Exclusion Criteria.

TABLE 8
Key Inclusion/Exclusion Criteria

| Inclusion | Exclusion |
|---|---|
| Male or female 18-65 years of age | History of seizure disorder, at risk of |
| DSM-5 criteria for current MDD | seizure, or any other condition that |
| without psychotic features | increases the risk of seizure |
| MADRS total score of ≥25 | Any current or recent medical, |
| Treated with at least one prior | psychiatric, or social condition that |
| antidepressant in the current major | was likely to interfere with the |
| depressive episode | conduct of the study, confound the |
| | interpretation of study results, or |
| | endangers the patient's well-being |

Table 9 shows the demographics and baseline characteristics.

TABLE 9
Demographics and Baseline Characteristics^{a}

| | DM-BUP (45 mg dextromethorphan - | |
|---|---|---|
| | 105 mg bupropion) (N = 145) | |
| Demographics | | |
| Age (years) | 45.6 | (13.07) |
| Female gender, n (%) | 88 | (60.7%) |
| Race, n (%) | | |
| White | 112 | (77.2%) |
| Black or African American | 25 | (17.2%) |
| Asian | 3 | (2.1%) |
| Other | 5 | (3.5%) |
| Clinical Characteristics | | |
| MADRS total score | 32.2 | (4.14) |
| CGI-S Score | 4.5 | (0.55) |
| SDS Score | 17.5 | (6.08) |

^{a}Data are mean (SD) unless otherwise stated.

Baseline disease severity represents a moderate-to-severely depressed population.

### Results

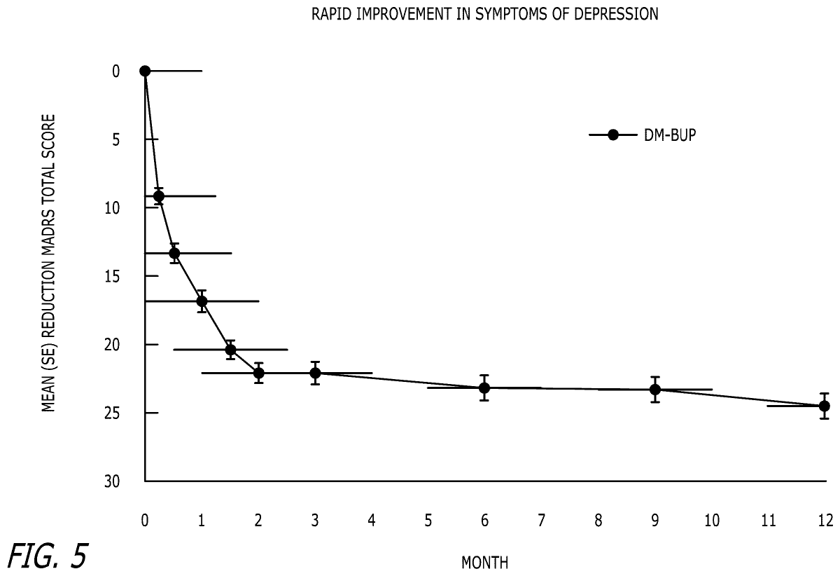

FIG. 5 shows rapid improvement in symptoms of depression.

Table 10 shows the mean improvement from baseline MADRS total score with Dextromethorphan-Bupropion treatment. As shown in Table 10, the following were observed.

Depressive symptoms improved rapidly after treatment with Dextromethorphan-Bupropion.

Mean change in MADRS total score from a baseline of 32.2 were: −9.1±7.64 (p<0.001), 13.3±8.58 (p<0.001), and −20.4±7.79 (p<0.001) points at Weeks 1, 2, and 6, respectively.

The improvement in depressive symptomatology was durable through 1 year.

TABLE 10
Mean Improvement from Baseline MADRS Total Score with Dextromethorphan-Bupropion Treatment

| | Week 1 | Week 2 | Week 4 | Week 6 | Month 2 | Month 3 | Month 4 | Month 9 | Month 12 |
|---|---|---|---|---|---|---|---|---|---|
| N | 141 | 136 | 131 | 124 | 118 | 112 | 95 | 91 | 83 |
| MADRS Change | −9.1 | −13.3 | −16.8 | −20.4 | −22.1 | −22.1 | −23.2 | −23.3 | −24.5 |
| from Baseline | | | | | | | | | |
| P-value | <0.001 | <0.001 | <0.001 | <0.001 | <0.001 | <0.001 | <0.001 | <0.001 | <0.001 |

### Clinical Response & Remission

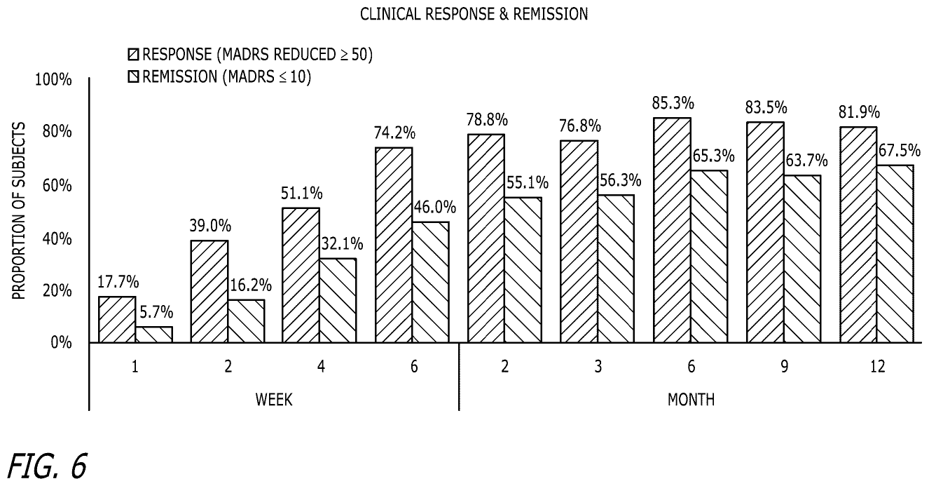

FIG. 6 shows the Clinical Response & Remission over time.

As shown in FIG. 6, the following were observed.

Clinical response (≥50% improvement) was achieved by 17.7% of patients at Week 1, 39.0% at Week 2, and 74.2% at Week 6.

Remission on the MADRS (≤10) was achieved by 5.7%, 16.2%, and 46.0% of patients at Weeks 1, 2, and 6, respectively.

Antidepressant effects were durable and sustained through Month 12.

CGI-S Scores Correlated Highly with MADRS Changes Over Time

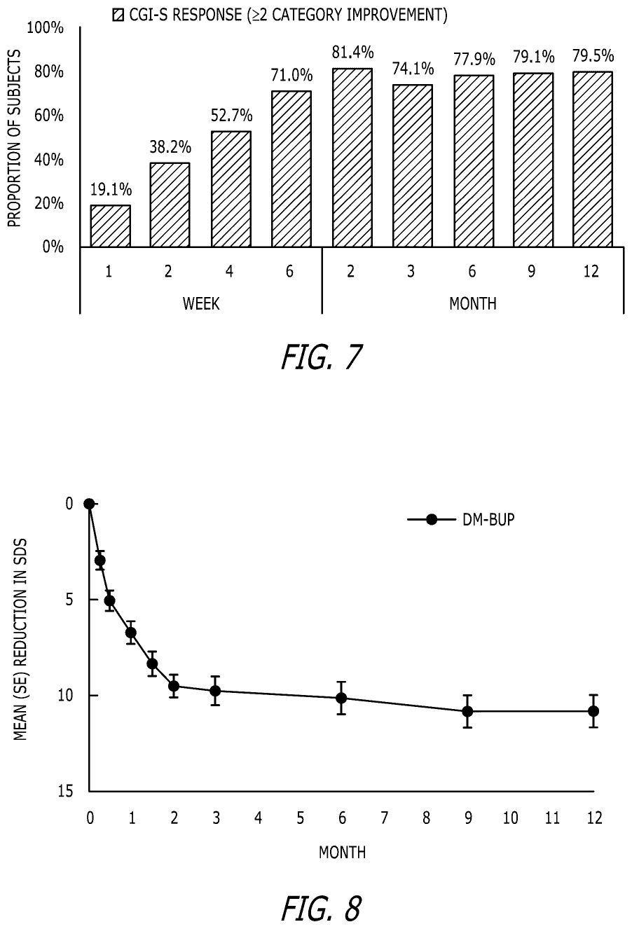

FIG. 7 shows the CGI-S response over time.

As shown in FIG. 7, the following were observed.

Response (≥2 category change) on the CGI-S was rapid, with 19.1% of patients responding at Week 1, 38.2% at Week 2, and 71.0% at Week 6.

The CGI-S improvement was durable, with response by 77.9% of patients at Month 6 and 79.5% at Month 12.

### Rapid Improvement in Function and SDS Remission

FIG. 8 shows the mean reduction in SDS over time.

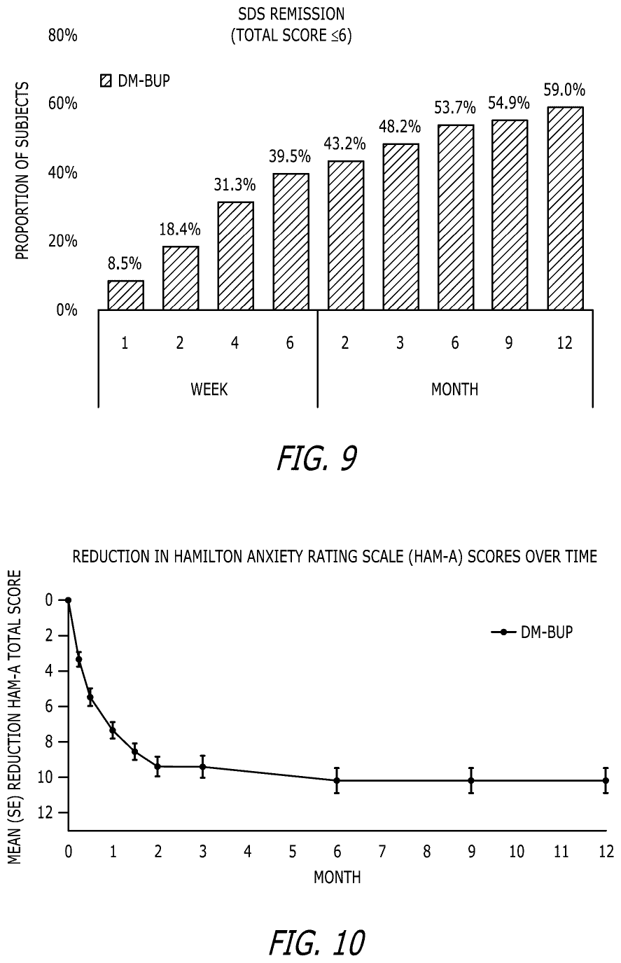

FIG. 9 shows the proportion of subjects in SDS remission over time.

The Baseline SDS score was 17.5±6.08. Mean reduction in SDS total score was 2.9±5.39, 5.0±5.78, and 8.3±6.71 points at Weeks 1, 2, and 6, respectively (p<0.001 for all) (FIG. 8).

Remission on the SDS (≤6) was achieved by 8.5% of patients at Week 1, 18.4% at Week 2, and 39.5% at Week 6 (FIG. 9).

Improvements on the SDS were durable and sustained through Month 12 (FIG. 9).

### Safety and Tolerability

Table 11 shows the adverse events (AE) in ≥5.0% of patients.

TABLE 11

| Adverse Events in ≥5.0% of patients | DM-BUP (N = 146) | |
|---|---|---|
| Any Adverse Event | 94 | (64.4%) |
| COVID-19 infection | 13 | (8.9%) |
| Nausea | 13 | (8.9%) |
| Headache | 11 | (7.5%) |
| Dry mouth | 9 | (6.2%) |
| Dizziness | 8 | (5.5%) |
| Insomnia | 8 | (5.5%) |

The long-term treatment with Dextromethorphan-Bupropion was well tolerated.

COVID-19 infection, nausea, and headache were the most common AEs (Table 11).

Discontinuation rate due to AEs was 8.9%.

Low rates of serious adverse events (5 patients; 3.4%) after long-term treatment with Dextromethorphan-Bupropion occurred; no SAE occurred in more than 1 patient.

### Conclusions

Dextromethorphan-Bupropion resulted in rapid and sustained improvement in depressive symptoms and functioning in patients who failed one prior antidepressant in the current major depressive episode.

High rates of clinical response and remission on the MADRS, and functional response on the SDS were achieved and maintained for up to 12 months of treatment.

These data provide further support for Dextromethorphan-Bupropion for the treatment of people with depression, including those who have received one prior treatment during their major depressive episode.

### Example 5

Improvement in Anxiety Symptoms in Depressed Patients Treated with Dextromethorphan-Bupropion: Results from the EVOLVE Open-Label, Long-Term Study

### Introduction

Major depressive disorder (MDD) is a serious disorder: MDD is a chronic, disabling, prevalent, biologically-based disorder, and a leading cause of suicide.

MDD is difficult to treat: In the largest open-label study conducted, STAR*D, only about ⅓ of individuals with MDD achieved remission with up to 12 weeks of therapy with the SSRI citalopram.

Anxiety in MDD: Anxiety has been reported in up to 50% of individuals with depression and has been associated with more difficult to treat depression.

Second line treatment: In STAR*D, following non-remission with an SSRI, remission rates for second line treatments were about 25% regardless of the switch strategy employed: switching to a different SSRI (sertraline), switching to a SNRI (venlafaxine), or switching to a NDRI (bupropion).

There is need for mechanistically novel approaches: The declining remission rates in STAR*D may be partially explained by the lack of pharmacological diversity amongst the different treatments, e.g., all antidepressants employed are thought to work in generally the same way: monoamine modulation.

Glutamatergic hypothesis of MDD: Clinical and preclinical evidence has implicated dysfunctional glutamatergic neurotransmission in the pathophysiology of MDD, suggesting a role for NMDA receptor antagonism in the treatment of MDD.

There is an urgent clinical need for: New, more effective, faster-acting, mechanistically novel, and well-tolerated MDD treatments.

### Objective

The objective is to evaluate the effects of Dextromethorphan-Bupropion (45 mg dextromethorphan HBr-105 mg bupropion HCl) on anxiety in MDD patients who had been treated with at least 1 prior antidepressant in the current major depressive episode.

### Study Design: EVOLVE

EVOLVE (Evaluation of NMDA Modulation for Depressive Episodes) was an open-label, US trial, in which patients were treated with Dextromethorphan-Bupropion twice daily for up to 15 months.

Eligible patients had either rolled in following completion of a prior Dextromethorphan-Bupropion study, or were directly enrolled.

All patients had a DSM-5 diagnosis of MDD, a MADRS score of ≥25, and had been treated with at least 1 prior antidepressant in the current major depressive episode.

A total of 186 patients were enrolled, consisting of 35 roll-over from a prior Dextromethorphan-Bupropion study (MERIT) and 146 directly enrolled. Here we present the results for the directly enrolled patients.

Newly enrolled subjects whose MADRS score did not decrease by ≥25% at Week 6 were discontinued from the study.

### Efficacy Outcome Measures:

1) Montgomery-Åsberg Depression Rating Scale (MADRS);
2) Hamilton Anxiety Rating Scale (HAM-A) scores; and
3) Sheehan Disability Scale (SDS).

### Statistical Analysis:

Efficacy analyses were conducted on the mITT population which consisted of all patients who received at least 1 dose of Dextromethorphan-Bupropion and provided at least 1 post-baseline efficacy measurement.

Safety analysis were conducted on the safety population, which included all patients who received at least 1 dose of Dextromethorphan-Bupropion.

Change from baseline P-values were analyzed using paired t-test.

Table 12 shows the key inclusion and exclusion criteria.

TABLE 12
Key Inclusion/Exclusion Criteria

| Inclusion | Exclusion |
|---|---|
| Male or female 18-65 years of age | History of seizure disorder, at risk of |
| DSM-5 criteria for current MDD | seizure, or any other condition that |
| without psychotic features | increases the risk of seizure |
| MADRS total score of ≥25 | Any current or recent medical, |
| Treated with at least one prior | psychiatric, or social condition that |
| antidepressant in the current major | was likely to interfere with the |
| depressive episode | conduct of the study, confound the |
| | interpretation of study results, or |
| | endangers the patient's well-being |

Demographics and Baseline Characteristics (mITT Population)

Table 13 shows the demographics and baseline characteristics (mITT population).

TABLE 13^{a}

| | DM-BUP (45 mg dextromethorphan - | |
|---|---|---|
| | 105 mg bupropion) (N = 145) | |
| Demographics | | |
| Age (years) | 45.6 | (13.07) |
| Female gender, n (%) | 88 | (60.7%) |
| Race, n (%) | | |
| White | 112 | (77.2%) |
| Black or African American | 25 | (17.2%) |
| Asian | 3 | (2.1%) |
| Other | 5 | (3.5%) |
| Clinical Characteristics | | |
| MADRS total score | 32.2 | (4.14) |
| CGI-S Score | 4.5 | (0.55) |
| HAM-A | 15.6 | (5.56) |

^{a}Data are mean (SD) unless otherwise stated.

Baseline depression severity represents a moderate-to-severely depressed population.

Baseline anxiety severity represents mild-to-moderate anxiety.

### Results

FIG. 10 and Table 14 show the reduction in Hamilton Anxiety Rating Scale (HAM-A) scores over time.

TABLE 14
HAM-A scores over time

| Week | Baseline | Week 1 | Week 2 | Week 4 | Week 6 | Month 2 | Month 3 | Month 6 | Month 9 | Month 12 |
|---|---|---|---|---|---|---|---|---|---|---|
| HAM-A score | 15.6 | 12.3 | 10.0 | 8.0 | 6.8 | 6.1 | 5.9 | 5.2 | 5.0 | 4.6 |
| P-value | | <0.001 | <0.001 | <0.001 | <0.001 | <0.001 | <0.001 | <0.001 | <0.001 | <0.001 |

As shown in FIG. 10 and Table 14, the following were observed:

Treatment with Dextromethorphan-Bupropion resulted in meaningful improvement in symptoms of anxiety.

Mean scores for HAM-A were <7 at Week 6, indicating remission of anxiety symptoms.

Reductions from baseline to Weeks 1, 2, and 6 were 3.4±5.34 (p<0.001), 5.5±5.81 (p<0.001), and 8.6±5.75 (p<0.001), respectively.

Improvements on the HAM-A were durable through Month 6 (−10.2±6.47; p<0.001) and Month 12 (−10.2±6.33; p<0.001).

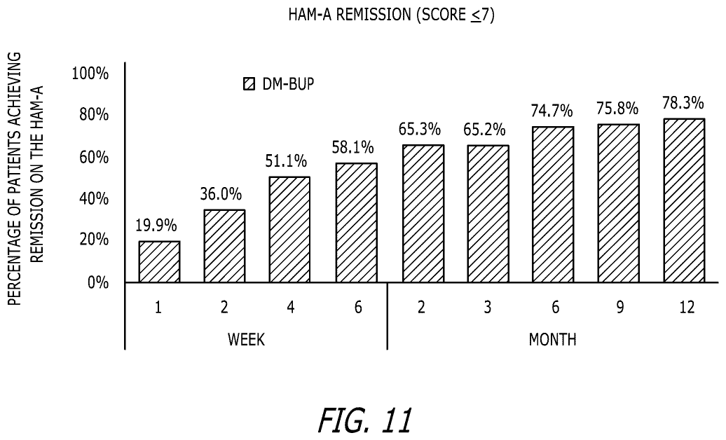

FIG. 11 shows the percent of patients achieving remissionon on the HAM-A (score≤7) over time.

As shown in FIG. 11, the following were observed:

Treatment with Dextromethorphan-Bupropion resulted in rapid rates of remission of anxiety.

By Week 4, over 50% of patients had achieved remission of anxiety symptoms.

Remission was durable, with 74.7% of patients remitting at Month 6 and 78.3% at Month 12.

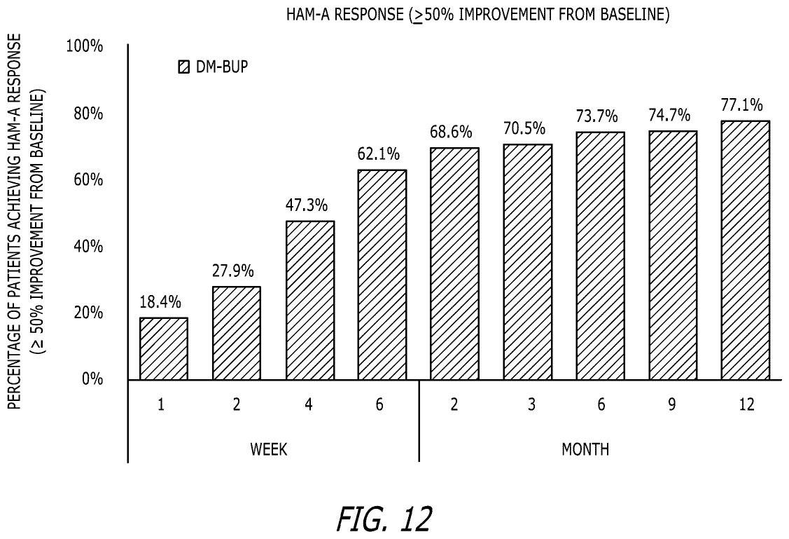

FIG. 12 show the percent of patients achieving HAM-A response (≥50% improvement from Baseline) over time.

As shown in FIG. 12, the following were observed:

Response on the HAM-A was achieved by 18.4%, 27.9%, and 62.1% of patients at Week 1, 2, and 6, respectively.

Response rates continued to improve through Month 6 (73.7%) and Month 12 (77.1%)

### Safety and Tolerability

Table 15 shows the adverse events (AEs) in ≥5% of patients.

TABLE 15

| Adverse Events in ≥5% of patients | DM-BUP (M = 146) | |
|---|---|---|
| Any Adverse Event | 94 | (64.4%) |
| COVID-19 infection | 13 | (8.9%) |
| Nausea | 13 | (8.9%) |
| Headache | 11 | (7.5%) |
| Dry mouth | 9 | (6.2%) |
| Dizziness | 8 | (5.5%) |
| Insomnia | 8 | (5.5%) |

Long-term treatment with Dextromethorphan-Bupropion was well tolerated.

COVID-19 infection, nausea, and headache were the most common AEs.

Discontinuation rate due to AEs was 8.9%.

Low rates of serious adverse events (5 patients; 3.4%) after long-term treatment with Dextromethorphan-Bupropion occurred. No SAE occurred in more than 1 patient.

### Conclusions

Treatment with Dextromethorphan-Bupropion reduced anxiety symptoms in patients with MDD.

Response and remission from anxiety symptoms were achieved as early as 1 week after starting treatment with Dextromethorphan-Bupropion.

These data are consistent with integrated data from Dextromethorphan-Bupropion controlled trials demonstrating reduction in the MADRS Inner Tension Item score versus control, at Week 1.

Long-term treatment with Dextromethorphan-Bupropion was generally safe and well tolerated.

These data provide additional evidence for the efficacy of Dextromethorphan-Bupropion in MDD including those with prior treatment and those with anxious features.

### Example 6

### Impact of Dextromethorphan-Bupropion, an Oral NMDA Receptor Antagonist, on Anhedonic Symptoms in Major Depressive Disorder

### Introduction

Major depressive disorder (MDD) is a serious disorder: MDD is a chronic, disabling, prevalent, biologically-based disorder, and a leading cause of suicide.

MDD is difficult to treat: 63% of MDD patients experience an inadequate response to current first-line oral therapies (STAR*D trial results), and the majority of these inadequate responders also fail second-line treatment (69%).

Anhedonia is one of the core features of major depressive disorder (MDD) and is present in up to 75% of individuals diagnosed with MDD.

Anhedonia is considered among the most bothersome aspects of MDD by patients, has been associated with decreased functioning and is a risk factor for non-response to antidepressant therapy.

Response to treatment takes time: Current oral antidepressants are associated with prolonged time to clinically meaningful response (up to 6-8 weeks).

Need for mechanistically novel approaches: Currently approved oral antidepressants work primarily through monoaminergic mechanisms.

Glutamatergic hypothesis of MDD: Clinical and preclinical evidence has implicated dysfunctional glutamatergic neurotransmission in the pathophysiology of MDD, suggesting a role for NMDA receptor antagonism in the treatment of MDD.

There is an urgent clinical need for: New, more effective, faster-acting, mechanistically novel, and well-tolerated MDD treatments.

### Objective

GEMINI was a phase 3, randomized, double-blind, placebo-controlled, multi-center, U.S. trial, in which 327 adult patients with confirmed moderate to severe MDD were randomized to either Dextromethorphan-Bupropion or placebo (NCT04019704).

A post-hoc analysis was conducted to determine the impact of Dextromethorphan-Bupropion as compared to placebo on the 5-item MADRS anhedonia subscale.

The objective is to evaluate the effect of Dextromethorphan-Bupropion as compared to placebo in improving anhedonic symptoms in MDD as assessed by the MADRS anhedonia subscale.

### Study Design: GEMINI

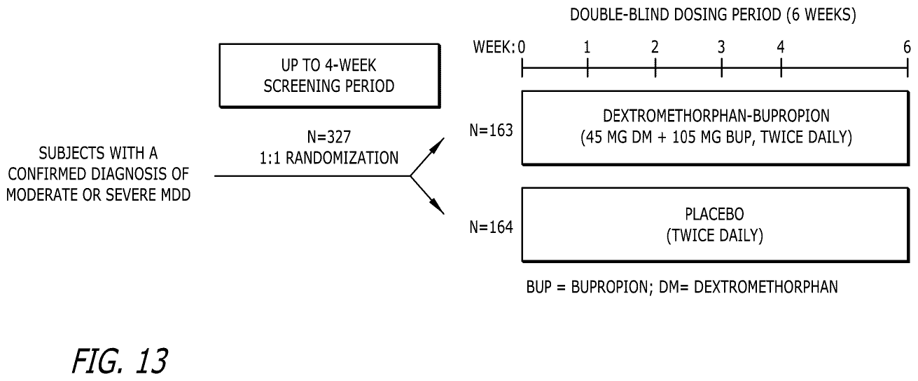

FIG. 13 shows the study design for GEMINI.

Primary Endpoint: Change from baseline in the Montgomery-Åsberg Depression Rating Scale (MADRS) total score at Week 6.

Key Secondary Endpoints: Change from baseline and in MADRS at Week 1 and Week 2.

MADRS Anhedonia Subscale: Change from baseline and rate of response as measured by the MADRS Anhedonia Subscale which includes 5-items:
1) Apparent sadness;
2) Reported sadness;
3) Concentration difficulties;
4) Lassitude; and
5) Inability to feel.

Previous research has demonstrated that the MADRS anhedonia subscale is highly correlated to the to the Snaith-Hamilton Pleasure Scale, a validated measure of hedonic tone.

Table 16 shows the key inclusion and exclusion criteria. Table 17 shows the demographics and baseline characteristics.

TABLE 16
Key Inclusion/Exclusion Criteria

| Inclusion | Exclusion |
|---|---|
| Male or female 18-65 years of | History of ECT, vagus nerve |
| age | stimulation, TMS or any experimental |
| DSM-5 criteria for current MDD | central nervous system treatment |
| without psychotic features | during the current episode or in the |
| MADRS total score of ≥25 | past 6 months |
| CGI-S score of ≥4 at baseline | Schizophrenia, bipolar disorder, |
| | obsessive compulsive disorder |
| | Psychiatric symptoms secondary to any |
| | other general medical condition |

TABLE 17
Demographics and Baseline Characteristics^{a}

| | |
|---|---|
| DM-BUP | Placebo |

| | | | | |
|---|---|---|---|---|
| Demographics | | | | |
| Age (years) | 42.1 | (12.71) | 41.1 | (13.78) |
| Female gender, n (%) | 98 | (60.1%) | 117 | (71.3%) |
| Race, n (%) | | | | |
| White | 88 | (54.0%) | 92 | (56.1%) |
| Black or African American | 61 | (37.4%) | 55 | (33.5%) |
| Clinical Characteristics | | | | |
| MADRS total score | 33.6 | (4.43) | 33.2 | (4.36) |
| MADRS Anhedonia score | 19.8 | (2.48) | 19.6 | (2.40) |
| CGI-S Score | 4.6 | (0.59) | 4.6 | (0.57) |

^{a}Data are mean (SD) unless otherwise stated.

Baseline disease severity represents a moderate-to-severely depressed population.

Demographics were similar across both Dextromethorphan-Bupropion and control groups.

### Results

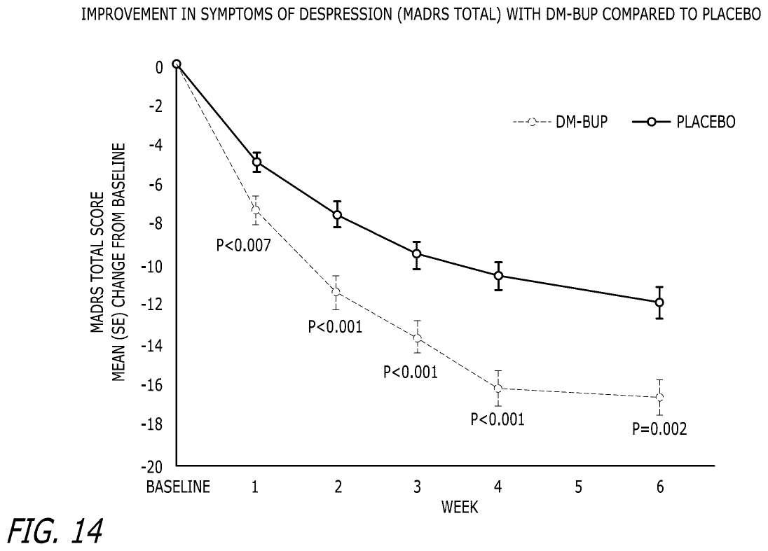

FIG. 14 shows the MADRS total score mean change from baseline with Dextromethorphan-Bupropion compared to placebo.

As shown in FIG. 14 and Table 18, the following were observed:
achieved the primary endpoint-statistically significant reduction from baseline on the MADRS total score at week 6 (−16.6 vs. −11.9, p=0.002), compared to placebo.
rapidly and statistically significantly reduced MADRS total score compared to placebo, by week 1, the first timepoint measured (p=0.007), at week 2 (p<0.001), and at all timepoints thereafter.

Table 18 shows the MADRS Total score change from baseline with at week 1 and week 2.

TABLE 18

| | DM-BUP | Placebo | | |
|---|---|---|---|---|
| Key Secondary Endpoints | (n = 156) | (n = 162) | Difference | P-Value |
| Change in MADRS Total | −7.3 | −4.9 | −2.4 | 0.007 |
| Score at Week 1 | | | | |
| Change in MADRS Total | −11.1 | −7.7 | −3.4 | <0.001 |
| Score at Week 2 | | | | |

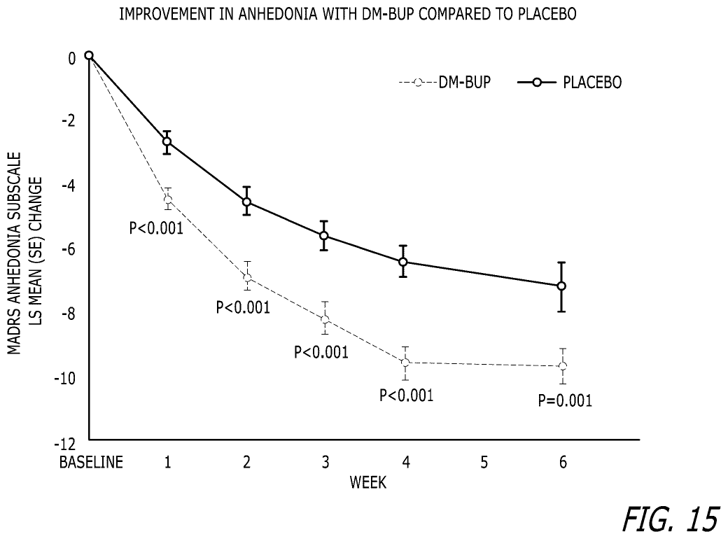

FIG. 15 shows the MADRS Anhedonia subscale LS mean change over time with Dextromethorphan-Bupropion compared to placebo.

As shown in FIG. 15, the following were observed:

At Week 1 (the first timepoint measured) treatment with Dextromethorphan-Bupropion resulted in a statistically significant mean reduction from baseline in the MADRS anhedonia subscale score of 4.44, versus 2.69 points for placebo (p<0.001).

By Week 6, the mean reduction from baseline in the MADRS anhedonia subscale was 9.70 for Dextromethorphan-Bupropion compared to 7.22 for placebo (p=0.001).

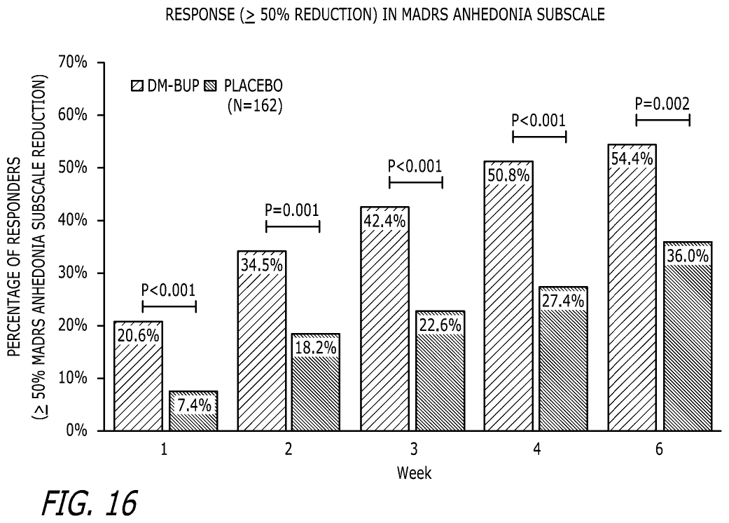

FIG. 16 shows the percentage of responders achieving ≥50% Reduction in MADRS Anhedonia Subscale.

As shown in FIG. 16, the rates of response (≥50% MADRS anhedonia subscale improvement) were statistically significantly greater for Dextromethorphan-Bupropion compared to placebo at Week 1 (p<0.001) and at every time point thereafter.

By week 6, 54% of Dextromethorphan-Bupropion patients were responders versus 36% of placebo patients at Week 6 (p=0.002).

### Safety and Tolerability

Table 19 shows the adverse event (AE) with Dextromethorphan-Bupropion compared with placebo.

TABLE 19

| | DM-BUP (N = 162) | | Placebo (N = 164) | |
|---|---|---|---|---|
| Any Treatment-emergent AE* | 100 | (61.7%) | 74 | (45.1%) |
| Dizziness | 26 | (16.0%) | 10 | (6.1%) |
| Nausea | 21 | (13.0%) | 14 | (8.5%) |
| Headache | 13 | (8.0%) | 6 | (3.7%) |
| Diarrhea | 11 | (6.8%) | 5 | (3.0%) |
| Somnolence | 11 | (6.8%) | 5 | (3.0%) |
| Dry mouth | 9 | (5.6%) | 4 | (2.4%) |

The most commonly reported AEs were dizziness, nausea, and headache.

Rates of discontinuation due to AEs were 6.2% for Dextromethorphan-Bupropion and 0.6%, for placebo.

### Conclusions

Dextromethorphan-Bupropion, a novel oral NMDA receptor antagonist, rapidly and statistically significantly improved depressive symptoms at Weeks 1 and 2.

Treatment with Dextromethorphan-Bupropion rapidly and significantly reduced anhedonic symptoms as well as overall depressive symptoms.

Dextromethorphan-Bupropion was generally safe and well-tolerated.

These data support the efficacy of Dextromethorphan-Bupropion in a broad range of symptomatology in patients with MDD.

### Example 7

Efficacy and Safety of Dextromethorphan-Bupropion (Dextromethorphan-Bupropion) in Patients with Major Depressive Disorder: A Phase 3 Randomized Clinical Trial (GEMINI)

Objective: Altered glutamatergic neurotransmission has been implicated in the pathogenesis of depression. This trial evaluated the efficacy and safety of dextromethorphan-bupropion, an oral N-methyl-D-aspartate (NMDA) receptor antagonist and 01 receptor agonist, in the treatment of major depressive disorder (MDD).

Methods: This double-blind, phase 3 trial, was conducted between June 2019 and December 2019. Patients with a DSM-5 diagnosis of MDD were randomized in a 1:1 ratio to receive dextromethorphan-bupropion (45 mg-105 mg tablet) or placebo, orally (once daily for days 1-3, twice daily thereafter) for 6 weeks. The primary endpoint was the change from baseline to week 6 in the Montgomery-Åsberg Depression Rating Scale (MADRS) total score. Other efficacy endpoints and variables included MADRS changes from baseline at week 1 and 2, clinical remission (MADRS score≤10), clinical response (≥50% reduction in MADRS score from baseline), clinician- and patient-rated global assessments, Quick Inventory of Depressive Symptomatology-Self-Rated, Sheehan Disability Scale, and quality of life measures.

Results: A total of 327 patients were randomized: 163 patients to dextromethorphan-bupropion and 164 patients to placebo. Mean baseline MADRS total scores were 33.6 and 33.2 in the dextromethorphan-bupropion and placebo groups, respectively. The least-squares mean change from baseline to week 6 in MADRS total score was −15.9 points in the dextromethorphan-bupropion group and −12.0 points in the placebo group (least-squares mean difference, −3.87; 95% confidence interval [CI], −1.39 to −6.36; P=0.002). Dextromethorphan-bupropion was superior to placebo for MADRS improvement at all time points including week 1 (P=0.007) and week 2 (P<0.001). Remission was achieved by 39.5% of patients with dextromethorphan-bupropion versus 17.3% with placebo (treatment difference, 22.2; 95% CI, 11.7 to 32.7; P<0.001), and clinical response by 54.0% versus 34.0%, respectively (treatment difference, 20.0%; 95% CI, 8.4%, 31.6%; P<0.001), at week 6. Results for most secondary endpoints were significantly better with dextromethorphan-bupropion than with placebo at almost all time points (e.g. CGI-S least-squares mean difference at week 6, −0.48; 95% CI, −0.48 to −0.79; P=0.002). The most common adverse events in the dextromethorphan-bupropion group were dizziness, nausea, headache, somnolence, and dry mouth. Dextromethorphan-bupropion was not associated with psychotomimetic effects, weight gain, or increased sexual dysfunction.

Conclusions: In this phase 3 trial in patients with MDD, treatment with dextromethorphan-bupropion resulted in significant improvements in depressive symptoms compared to placebo starting 1 week after treatment initiation and was generally well tolerated.

Trial Registration: ClinicalTrials.gov Identifier: NCT04019704

Major depressive disorder (MDD) is a prevalent, disabling, chronic, biologically based disorder, which impairs social, occupational, and educational functioning. It is the leading cause of disability worldwide and is associated with increased suicide risk, morbidity, and mortality. Currently approved oral antidepressants work primarily via monoamine pathways. Partial or inadequate response is common with these agents, and they typically take several weeks to produce clinically meaningful effects. In the Sequenced Treatment Alternatives to Relieve Depression (STAR*D) trial, about two thirds of depression patients failed to achieve remission with first-line treatment, and of those who experienced a clinical response, approximately 60% did so only at or after 8 weeks of treatment.

Involvement of the glutamatergic system in the pathogenesis of depression is suggested by data from neuroimaging, cellular, and clinical studies. This evidence includes findings of abnormal glutamate levels in the cortex of depressed patients using magnetic resonance spectroscopy, observations of abnormal N-methyl-D-aspartate (NMDA) receptor expression and signaling in postmortem cortical preparations from depressed patients, and demonstration of antidepressant efficacy in studies with parenteral administration of the NMDA receptor antagonist ketamine.

Dextromethorphan is an uncompetitive antagonist of the NMDA receptor (an ionotropic glutamate receptor) and a 01 receptor agonist. Blockade of the NMDA receptor and agonism of the 01 receptor modulate glutamate signaling in the central nervous system. The clinical utility of dextromethorphan has been limited in humans by its rapid and extensive metabolism through cytochrome P450 (CYP) 2D6 yielding subtherapeutic plasma levels. A tablet combining dextromethorphan and bupropion (hereafter, dextromethorphan-bupropion) has been formulated to increase the bioavailability and half-life of dextromethorphan and has been developed for the treatment of MDD. The bupropion component serves to increase plasma dextromethorphan concentrations by inhibiting its metabolism. Breakthrough Therapy designation was granted by the US Food and Drug Administration for dextromethorphan-bupropion for the treatment of MDD in March 2019. The objective of this phase 3 trial was to assess the efficacy and safety of dextromethorphan-bupropion compared to placebo in the treatment of patients with MDD.

### Clinical Points

Currently available oral antidepressants work primarily via the monoamine pathway, may be associated with partial or inadequate response, and typically take several weeks to produce clinically meaningful effects.

In this large randomized controlled trial, dextromethorphan-bupropion, an orally administered NMDA receptor antagonist and σ1 receptor agonist, rapidly reduced depressive symptoms and induced remission in patients with major depressive disorder. Dextromethorphan-Bupropion was well tolerated.

### Methods

### Trial Design and Oversight

The GEMINI (Glutamatergic and Monoaminergic Modulation in Depression) study was a phase 3, randomized, double-blind, placebo-controlled, 6-week trial conducted at 40 centers in the United States from June 2019 to December 2019. The trial was conducted in accordance with the International Council on Harmonization guidelines for Good Clinical Practice and the principles of the Declaration of Helsinki. The site investigators gathered the trial data, and the sponsor ensured that all persons administering rating scales were qualified and appropriately trained. All sites gained independent review board approval, and all patients provided written informed consent prior to participation.

### Patient Population

Patients were men or women 18-65 years of age with a primary diagnosis of MDD, experiencing a major depressive episode of at least 4 weeks in duration, and having a Montgomery-Åsberg Depression Rating Scale (MADRS) total score of 25 or higher, corresponding to moderate or greater severity, with higher scores indicating more severe depression. Patients were also required to have a score on the Clinician Global Impression-Severity (CGI-S) scale of 4 or higher (range, 1 to 7, with higher scores indicating greater severity of illness). The diagnosis of depression was established using the Diagnostic and Statistical Manual of Mental Disorders, Fifth Edition (DSM-5), criteria for MDD without psychotic features, based on the Structured Clinical Interview, which has been shown to diagnose MDD more conservatively than other structured interviews. As part of the screening process, an independent assessor confirmed the eligibility and symptom severity of each patient. The assessment consisted of a clinical review of all available documentation including complete medical history, and clinician- and patient-reported outcome measures.

Key exclusion criteria included bipolar disorder, psychotic disorder, panic disorder, obsessive-compulsive disorder, treatment-resistant depression (defined as ≥2 adequate failed antidepressant treatments in the current major depressive episode), alcohol/substance use disorder within the past year, clinically significant risk of suicide, and history of seizure disorder.

### Trial Design and Procedures

Eligible patients were randomly assigned in a 1:1 ratio to receive either dextromethorphan-bupropion (45 mg-105 mg) or placebo orally for 6 weeks. Randomization was performed by a central interactive web response system. Study medication was provided by the trial sponsor and was identical in form and appearance; all investigators, patients, and study personnel involved in the study were blinded to study treatment. Patients received their assigned study medication once daily for 3 days, then twice daily thereafter. Study visits occurred at 1, 2, 3, 4, and 6 weeks after the baseline visit. A safety follow-up visit occurred at week 7, 1 week following the last dose of study medication. There were no formal discontinuation criteria; however, patients were free to withdraw consent for any reason and investigators were free to remove a patient from study for any safety-related reason. The dose of dextromethorphan-bupropion, titrated to twice daily, was selected based on the results of pharmacokinetic trials. The clinical trial was listed on ClinicalTrials.gov (NCT04019704).

### Endpoints

The primary endpoint was the change from baseline to week 6 in the MADRS total score. The MADRS is a 10-item clinician-rated questionnaire ranging from 0 to 60, with higher scores representing more severe depression. The key secondary endpoints were the change from baseline in the MADRS total score at week 1; change from baseline in the MADRS total score at week 2; remission, defined as MADRS total score≤10, at week 2; and clinical response, defined as ≥50% reduction in MADRS total score, at week 6.

Other secondary endpoints included the Clinician Global Impression-Improvement (CGI-I; scores range from 1 [very much improved] to 7 [very much worse])¹⁹; CGI-S (scores range from 1 [normal state] to 7 [among the most extremely ill]); Patient Global Impression-Improvement (PGI-I; scores range from 1 [very much improved] to 7 [very much worse]); Quick Inventory of Depressive Symptomatology-Self-Rated (QIDS-SR-16; scores range from 0 to 27 with higher scores representing more severe depression); Sheehan Disability Scale (SDS; scores range from 0 to 30 with higher scores indicating more severe disability); the Quality of Life Enjoyment and Satisfaction Questionnaire-Short Form (Q-LES-Q-SF; scores are based on the percentage of the maximum total score with higher percentages indicating greater satisfaction); and the MADRS-6 (a subscale of the 10-item MADRS evaluating the core symptoms of depression [apparent sadness, reported sadness, inner tension, lassitude, inability to feel, and pessimistic thought]).

Safety was assessed based on the incidence of adverse events; changes in vital signs, clinical laboratory measurements, physical examinations, and electrocardiograms; assessment of suicidal ideation and behavior, with the use of the Columbia-Suicide Severity Rating Scale (C-SSRS); and assessment for withdrawal-related symptoms, using the Physician Withdrawal Checklist. Adverse events during the treatment period were defined as adverse events occurring from the time of administration of the first dose of dextromethorphan-bupropion or placebo until 7 days after the last dose.

### Statistical Analysis

The safety analysis set included all patients who received at least 1 dose of study medication. Efficacy analyses were performed on the modified intent-to-treat population, which consisted of all patients who were randomized, received at least 1 dose of study medication, and had at least 1 post-baseline efficacy assessment. The primary efficacy variable, change from baseline in MADRS, was analyzed using a mixed model for repeated measures. This analysis of covariance mixed-effect model for repeated measures included treatment, week, and treatment-by-week interaction as factors, baseline value as a covariate, and patient as a random effect. All other change from baseline efficacy variables were analyzed using this method. Treatment effects and treatment differences at each time point were estimated using the least squares mean estimates. Efficacy variables related to percentages (e.g., clinical response and remission rates, CGI-I, PGI-I) were performed on observed cases and analyzed via χ² tests. Analyses were performed using SAS Version 9.4, and all hypothesis tests were conducted at a 2-sided a level of 0.05.

If results were found to be positive on the MADRS primary endpoint, then other analyses (response, remission) were to be performed on this variable to examine clinical relevance. To adjust for multiplicity, if the primary endpoint was established, then key secondary endpoints were examined with the use of hierarchical hypothesis tests in a prespecified fixed-sequence procedure with the following order (we would stop after reaching a nonsignificant result): change in MADRS total score from baseline to week 2; percentage of patients achieving remission, defined as MADRS total score of ≤10, at week 2; change in MADRS total score from baseline to week 1; percentage of patients achieving clinical response, defined as ≥50% reduction in MADRS total score, at week 6; CGI-I at week 6; change in CGI-S from baseline to week 6; CGI-I at week 1; percentage of patients achieving remission at week 1; and change in SDS from baseline to week 6. For all other secondary endpoints, no adjustment for multiplicity was performed and nominal P values for these are presented.

There was no imputation of missing data for primary or secondary endpoints. Sensitivity analyses for the primary efficacy variable were performed based on the random replacement and tipping point methods.

A sample size of approximately 150 patients per treatment group would provide 90% power to detect a treatment difference on the primary efficacy variable of change in MADRS, at a 2-sided significance level of 0.05, assuming an effect size of 0.31.

### Results

### Patient Characteristics

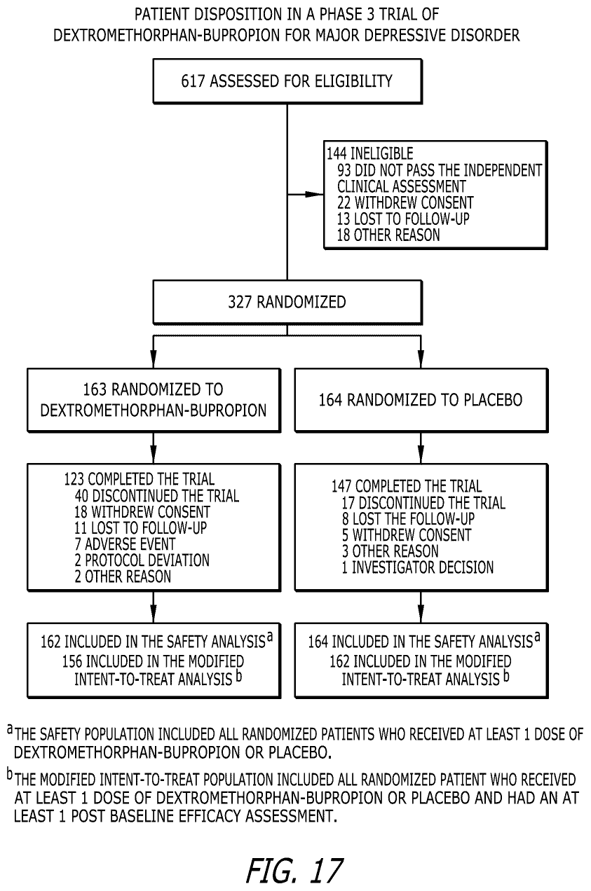

A total of 617 patients were screened, of whom 327 were randomly assigned in a 1:1 ratio to receive dextromethorphan-bupropion (163 patients) or placebo (164 patients) (FIG. 17). The modified intent-to-treat population consisted of 156 patients in the dextromethorphan-bupropion group and 162 patients in the placebo group. The demographics and clinical characteristics at baseline were generally similar for the 2 trial groups (Table 20). The dextromethorphan-bupropion group included more men than the placebo group (39.1% vs 27.8%; P=0.033). At baseline, mean MADRS total scores were 33.6 in the dextromethorphan-bupropion group and 33.2 in the placebo group, and mean CGI-S scores were 4.6 in both groups. The number of patients who completed the trial was 123 in the dextromethorphan-bupropion group and 147 in the placebo group.

FIG. 17 shows the patient disposition in a phase 3 trial of Dextromethorphan-Bupropion for major depressive disorder.

TABLE 20
Baseline Characteristics in a Phase 3 Trial of Dextromethorphan-
Bupropion for Major Depressive Disorder (mITT)

| | Treatment Group | | | |
|---|---|---|---|---|
| | AXS-05 | | | |
| | (Dextromethorphan- | | | |
| | Bupropion) | | Placebo | |
| Characteristic | (N = 156) | | (N = 162) | |
| Age, y | | | | |
| Mean (SD) | 421 | (12.80) | 41.2 | (13.77) |
| Range | 18-64 | | 18-65 | |
| Sex, n (%) | | | | |
| Female | 95 | (60.9) | 117 | (72.2) |
| Male | 61 | (39.1)^{a} | 45 | (27.8) |
| Race, n(%) | | | | |
| White | 84 | (53.8) | 92 | (56.8) |
| Black or African-American | 58 | (37.2) | 54 | (33.3) |
| Asian | 9 | (5.8) | 8 | (4.9) |
| Multiple | 3 | (1.9) | 2 | (1.2) |
| Other | 2 | (1.3) | 6 | (3.7) |
| BMI (kg/m²) | | | | |
| Mean (SD) | 29.3 | (5.61) | 29.3 | (5.69) |
| Range | 18.2-39.8 | | 18.1-39.7 | |
| MADRS total score^{b} | | | | |
| Mean (SD) | 33.6 | (4.43) | 33.2 | (4.36) |
| Range | 26.0-48.0 | | 25.0-46.0 | |
| CGI-S score^{c} | | | | |
| Mean (SD) | 4.6 | (0.59) | 4.6 | (0.57) |

| | | |
|---|---|---|
| Range | 4.0-6.0 | 4.0-6.0 |

^{a}Significantly more males were enrolled into the dextromethorphan-bupropion group compared to the placebo group (P = 0.033).
^{b}scores on the MADRS range from 0 to 60, with higher scores indicating more severe depression.
^{c}scores on the CGI-S range from 1 to 7, with higher scores representing more severe disease.
Abbreviations: BMI = body mass index, CGI-S = Clinical Global Impression-Severity, MADRS = Montgomery-Åsberg Depression Rating Scale, SD = standard deviation.

### Efficacy

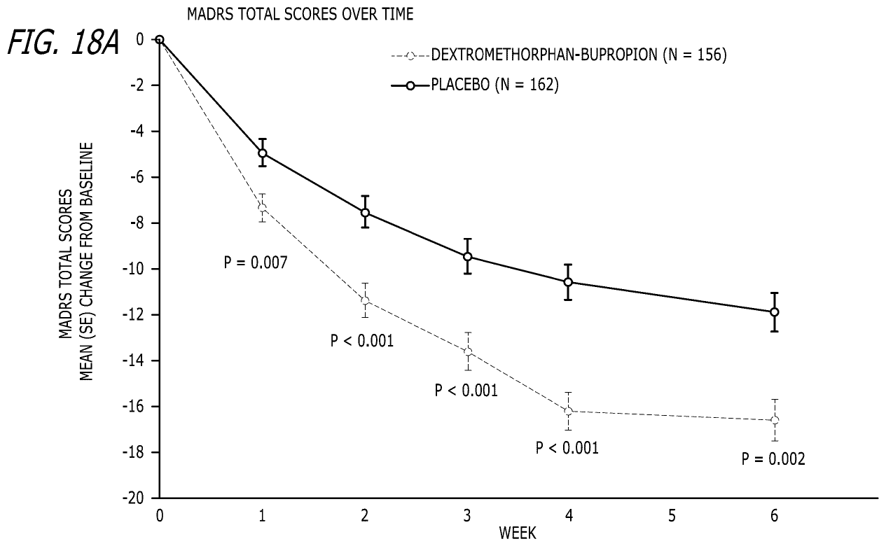

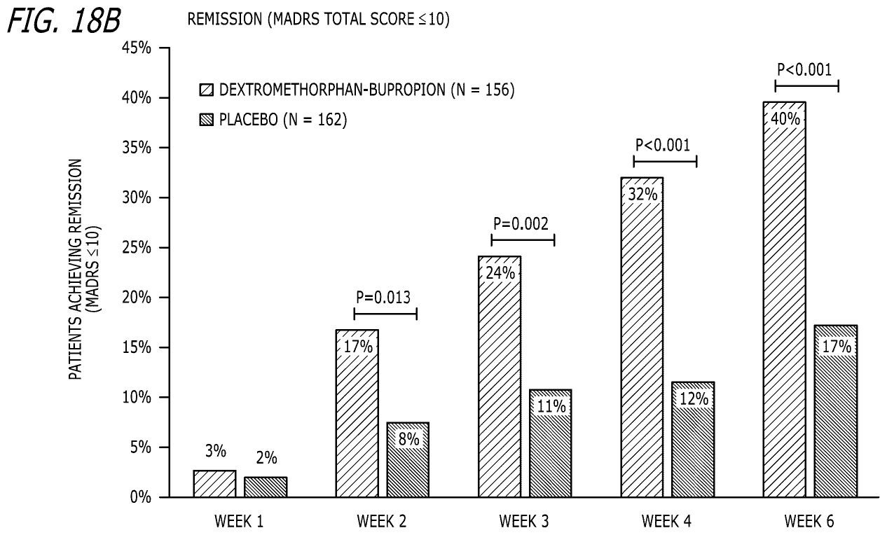

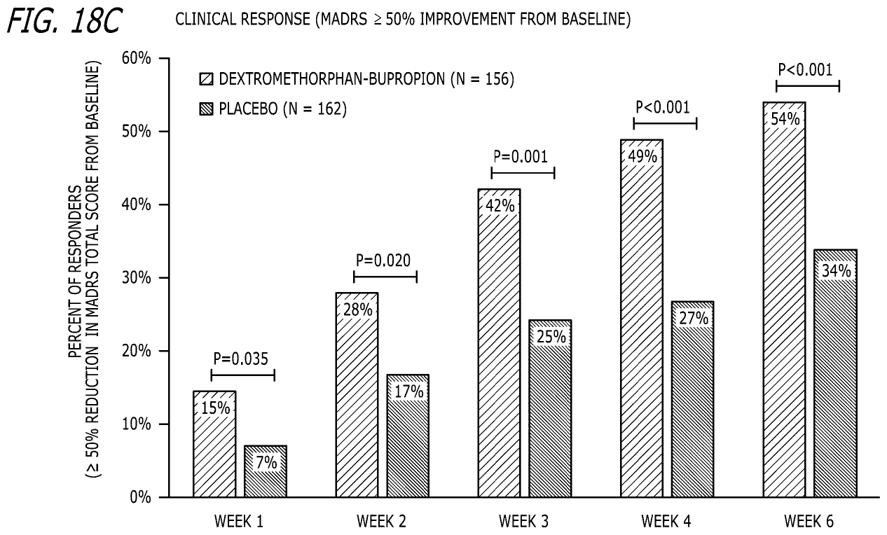

Dextromethorphan-bupropion significantly reduced MADRS total scores compared to placebo at all time points assessed (FIGS. 18A, 18B and 18C). The least-squares mean change from baseline to week 6 in MADRS total score was −15.9 points in the dextromethorphan-bupropion group and −12.0 points in the placebo group (least-squares mean difference, −3.87; 95% confidence interval [CI], −1.39 to −6.36; P=0.002). Results of the sensitivity analyses of the change in MADRS total score from baseline to week 6 were also statistically significant, favoring dextromethorphan-bupropion with a similar magnitude of treatment difference as the primary analysis. At week 1, the first time point, the least-squares mean change from baseline in MADRS total score was −7.20 points in the dextromethorphan-bupropion group and −4.97 points in the placebo group (least-squares mean difference, −2.23; 95% CI, −0.60 to −3.86; P=0.007). At week 2, the least-squares mean change from baseline in MADRS total score was −11.09 points in the dextromethorphan-bupropion group and −7.66 points in the placebo group (least-squares mean difference, −3.44; 95% CI, −1.40 to −5.47; P<0.001).

FIG. 18A displays the change from baseline in the MADRS total score over time. Scores on the MADRS range from 0 to 60, with higher scores indicating more severe depression. The results are presented as means, and the error bars represent standard errors. P values are calculated based on least squares means. FIG. 18B displays the percentage of patients achieving remission (MADRS total score≤10) over time. P values are calculated via X² tests. FIG. 18C displays the percentage of patients achieving clinical response (>50% reduction from baseline in MADRS total score) over time. P values are calculated via X² tests. MADRS represents Montgomery-Åsberg Depression Rating Scale, and SE represents standard error.

Remission, defined as a MADRS total score of ≤10, was achieved by a significantly greater percentage of patients in the dextromethorphan-bupropion group than in the placebo group at week 2 (16.9% and 7.5%, respectively; treatment difference, 9.4%; 95% CI, 1.9%-16.8%; P=0.013) and at every time point thereafter. At week 6, the percentage of patients achieving remission was 39.5% in the dextromethorphan-bupropion group and 17.3% in the placebo group (treatment difference, 22.2%; 95% CI, 11.7%-32.7%; P<0.001). Clinical response, defined as a ≥50% reduction in MADRS total score, was achieved by a significantly greater percentage of patients in the dextromethorphan-bupropion group than in the placebo group at all time points. At week 6, the percentage of patients achieving clinical response was 54.0% in the dextromethorphan-bupropion group and 34.0% in the placebo group (treatment difference, 20.0%; 95% CI, 8.4%-31.6%; P<0.001) (Table 21).

TABLE 21
Primary and Secondary Outcomes in a Phase 3 Trial of Dextromethorphan-
Bupropion for Major Depressive Disorder (mITT)^{a,b}

AXS-05
Bupropion)
(Dextromethorphan-

| | | | | |
|---|---|---|---|---|
| | Bupropion) | Placebo | Difference | P |
| Outcome | (N = 156) | (N = 162) | (95% CI) | Value |

Primary Endpoint
MADRS total score change from baseline

| | | | | | | | |
|---|---|---|---|---|---|---|---|
| Week 6 LS mean (SE) | −15.9 | (0.9) | −12.0 | (0.9) | −3.9 | (−1.4 to −6.4) | .002 |

Secondary Outcomes (Clinician Rated)
MADRS total score change from baseline

| | | | | | | | |
|---|---|---|---|---|---|---|---|
| Week 1 LS mean (SE) | −7.2 | (0.6) | −5.0 | (0.6) | −2.2 | (−0.6 to −3.9) | .007 |
| Week 2 LS mean (SE) | −11.1 | (0.7) | −7.7 | (0.7) | −3.4 | (−1.4 to −55) | <.001 |

Clinical response (MADRS ≥50%)

| | | | | | | | |
|---|---|---|---|---|---|---|---|
| Week 1 Responder, n (%) | 23 | (14.8%) | 12 | (7.4%) | 7.4% | (0.5% to 14.3%) | .035 |
| Week 2 Responder, n (%) | 40 | (28.2%) | 27 | (17.0%) | 11.2% | (1.8% to 20.6%) | .020 |
| Week 6 Responder, n (%) | 67 | (54.0%) | 51 | (34.0%) | 20.0% | (8.4% to 31.6%) | <.001 |

Remission (MADRS ≤10)

| | | | | | | | |
|---|---|---|---|---|---|---|---|
| Week 1 Remitter, n (%) | 4 | (2.6%) | 3 | (1.9%) | 0.7% | (−2.5% to 4.0%) | .659 |
| Week 2 Remitter, n (%) | 24 | (16.9%) | 12 | (7.5%) | 9.4% | (1.9% to 16.8%) | .013 |
| Week 6 Remitter, n (%) | 49 | (39.5%) | 26 | (17.3%) | 22.2% | (11.7% to 32.7%) | <.001 |

CGI-I (% patients)

| | | | | | | | |
|---|---|---|---|---|---|---|---|
| Week 1 Marked/moderate | 34 | (21.9%) | 21 | (13.0%) | 9.0% | (0.7% to 17.3%) | 0.035 |
| improvement, n (%) | | | | | | | |
| Week 2 Marked/moderate | 62 | (43.7%) | 35 | (22.0%) | 21.6% | (11.3% to 32.0%) | <.001 |
| improvement, n (%) | | | | | | | |
| Week 6 Marked/moderate | 72 | (57.6%) | 64 | (43.0%) | 14.6% | (2.9% to 26.4%) | .016 |
| improvement, n (%) | | | | | | | |

CGI-S score change from baseline

| | | | | | | | |
|---|---|---|---|---|---|---|---|
| Week 6 LS mean (SE) | −1.7 | (0.1) | −1.2 | (0.1) | −0.5 | (−0.2 to −0.8) | .002 |

MADRS-6 change from baseline

| | | | | | | | |
|---|---|---|---|---|---|---|---|
| Week 6 LS mean (SE) | −11.2 | (0.6) | −8.5 | (0.6) | −2.8 | (−1.0 to −4.5) | .002° |

Secondary Outcomes (Patient Reported)
QIDS-SR-16 change from baseline

| | | | | | | | |
|---|---|---|---|---|---|---|---|
| Week 6 LS mean (SE) | −7.7 | (0.5) | −5.7 | (0.4) | −2.0 | (−0.8 to −3.3) | .001° |

PGI-I (% patients)

| | | | | | | | |
|---|---|---|---|---|---|---|---|
| Week 6 Very much/much | 59 | (47.2%) | 47 | (31.3%) | 15.9% | (4.4% to 27.3%) | .007° |
| improved, n (%) | | | | | | | |

Q-LES-Q-SF change from baseline

| | | | | | | | |
|---|---|---|---|---|---|---|---|
| Week 6 LS mean (SE) | 19.8 | (1.6) | 14.4 | (1.5) | 5.5 | (1.3 to 9.7) | .011° |

SDS total score change from baseline

| | | | | | | | |
|---|---|---|---|---|---|---|---|
| Week 6 LS mean (SE) | 9.0 | (0.7) | 6.3 | (0.6) | 2.7 | (1.0 to 4.5) | .002° |

^{a}Change from baseline variables were analyzed using a mixed model for repeated measures. This analysis of covariance mixed-effect model for repeated measures included treatment, week, and treatment-by-week interaction as factors, baseline value as a covariate, and patient as a random effect. Treatment effects and treatment differences at each timepoint were estimated using the least-squares mean estimates. Efficacy variables related to percentages were analyzed via X² tests. All hypothesis tests were conducted at a 2-sided α level of 0.05.
^{b}The MADRS is a 10-item clinician-rated questionnaire ranging from 0 to 60 with higher scores representing more severe depression. CGI-I scores range from 1 (very much improved) to 7 (very much worse). CGI-S scores range from 1 (normal state) to 7 (among the most extremely ill). MADRS-6 is a subscale of the lo-item MADRS evaluating the core symptoms of depression (apparent sadness, reported sadness, inner tension, lassitude, inability to feel, and pessimistic thought). QIDS-SR-16 scores range from 0 to 27 with higher scores representing more severe depression. PGI-I scores range from 1 (very much improved) to 7 (very much worse). Q-LES-Q-SF scores are based on the percentage of the maximum total score with higher percentages indicating greater satisfaction. SDS scores range from 0 to 30 with higher scores indicating more severe disability.
^{c}Nominal P value.
Abbreviations: CGI-I = Clinical Global Impression-Improvement, CGI-S = Clinical Global Impression-Severity, CI = confidence interval, MADRS-6 = 6-Item Montgomery-Åsberg Depression Rating Scale, LS mean = least-squares mean, PGI-I = Patient Global Impression-Improvement, QIDS-SR-16 = Quick Inventory of Depressive Symptomatology-Self-Rated, Q-LES-Q-SF = Quality of Life Enjoyment and Satisfaction Questionnaire-Short Form, SDS = Sheehan Disability Scale, SE = standard error.

Marked or moderate improvement on the CGI-I was achieved by a significantly higher percentage of patients in the dextromethorphan-bupropion group than in the placebo group at all time points assessed (Table 21). The percentage of patients achieving marked or moderate improvement on the CGI-I at week 6 was 57.6% in the dextromethorphan-bupropion group and 43.0% in the placebo group (treatment difference, 14.6%; 95% CI, 2.9%-26.4%; P=0.016). Dextromethorphan-bupropion significantly reduced CGI-S scores compared to placebo at all time points assessed. The least-squares mean change from baseline to week 6 in CGI-S score was −1.69 points in the dextromethorphan-bupropion group and −1.29 points in the placebo group (least-squares mean difference, −0.48; 95% CI, −0.48 to −0.79; P=0.002).

At all the time points tested, least-squares mean improvements from baseline on the QIDS-SR-16, the MADRS-6 subscale, and the Q-LES-Q-SF were statistically significantly greater, and the percentage of patients much or very much improved on the PGI-I statistically significantly higher, in the dextromethorphan-bupropion group than in the placebo group. Least-squares mean improvement from baseline on the SDS was statistically significantly greater in the dextromethorphan-bupropion group than in the placebo group at week 2 and at every time point thereafter (Table 21). P values for these endpoints are nominal because of the lack of adjustment for multiplicity for these outcomes.

### Safety

The percentage of patients in whom adverse events occurred during the treatment period was 61.7% in the dextromethorphan-bupropion group and 45.1% in the placebo group. The most common adverse events in the dextromethorphan-bupropion group were dizziness, nausea, headache, somnolence, and dry mouth. There was 1 serious adverse event in the trial, a case of pancreatitis occurring in the dextromethorphan-bupropion group that was determined not related to study medication by the investigator. One adverse event rated severe (migraine) was observed in the dextromethorphan-bupropion group, while 2 events rated severe (back pain, finger fracture repair) were reported in the placebo group. Adverse events resulting in discontinuation of study medication occurred in 6.2% of patients in the dextromethorphan-bupropion group and 0.6% of patients in the placebo group. Dextromethorphan-bupropion was not associated with psychotomimetic effects, weight gain, or increased sexual dysfunction (Table 22). There were no suicide-related adverse events or suicidal behaviors on the C-SSRS in either treatment group. One patient in each treatment group reported suicidal ideation without intent on the C-SSRS at week 6. There were no signals of withdrawal after discontinuation of dextromethorphan-bupropion.

TABLE 22
Summary of Adverse Events in a Phase 3 Trial of Dextromethorphan-
Bupropion for Major Depressive Disorder (Safety Population^{a})

| | Dextromethorphan- | | | |
|---|---|---|---|---|
| | Bupropion | | Placebo | |
| | (N = 162) | | (N = 164) | |
| Any adverse event, n (%) | 100 | (61.7%) | 74 | (45.1%) |
| Serious^{b} adverse events, n (%) | 1 | (0.6%) | 0 | (0.0%) |
| Severe^{c} adverse events, n (%) | 1 | (0.6%) | 2 | (1.2%) |
| Adverse event leading to | 10 | (6.2%) | 1 | (0.6%) |
| discontinuation of study drug, | | | | |
| n (%) | | | | |
| Adverse events occurring | | | | |
| in ≥3% of patients in the | | | | |
| dextromethorphan-bupropion | | | | |
| group, n (%) | | | | |
| Dizziness | 26 | (16.0%) | 10 | (6.1%) |
| Nausea | 21 | (13.0%) | 14 | (8.5%) |
| Headache | 13 | (8.0%) | 6 | (3.7%) |
| Diarrhea | 11 | (6.8%) | 5 | (3.0%) |
| Somnolence | 11 | (6.8%) | 5 | (3.0%) |
| Dry mouth | 9 | (5.6%) | 4 | (2.4%) |
| Hyperhidrosis | 8 | (4.9%) | 0 | |
| Anxiety | 7 | (4.3%) | 2 | (1.2%) |
| Constipation | 6 | (3.7%) | 3 | (1.8%) |
| Decreased appetite | 6 | (3.7%) | 1 | (0.6%) |
| Insomnia | 6 | (3.7%) | 3 | (1.8%) |
| Upper respiratory tract infection | 5 | (3.1%) | 5 | (3.0%) |
| Change from baseline in body | −0.2 | (±7.8) | −0.4 | (±2.3) |
| weight at week 6, mean (SD), kg | | | | |

^{a}The safety population included all randomized patients who received at least I dose of dextromethorphan-bupropion or placebo. Adverse events that occurred during the treatment period were defined as those that started or worsened from the time of the first dose of dextromethorphan-bupropion or placebo through 7 days after the last dose.
^{b}A serious adverse event was defined as any adverse event that resulted in death, was immediately life-threatening, led to inpatient hospitalization or prolongation of hospitalization, or caused persistent or clinically significant disability or incapacity.
^{c}A severe adverse event was defined as any event that interrupted usual activities of daily living, or significantly affected clinical status, or requires intensive therapeutic intervention.

### Discussion

In this randomized, r placebo-controlled trial, dextromethorphan-bupropion demonstrated statistically significant antidepressant efficacy on the primary endpoint and the majority of secondary endpoints across multiple symptom-specific and global measures. Treatment with dextromethorphan-bupropion resulted in statistically significantly greater reductions in MADRS total score than placebo starting at week 1 and continuing at every time point thereafter. The medication-placebo difference for dextromethorphan-bupropion on the change in MADRS was substantial at all time points, being approximately 2-3 points at weeks 1 and 2 and increasing to approximately 4-5 points at weeks 4 and 6. This treatment effect compares favorably to the approximately 2.5-point mean difference from placebo seen at 6 to 8 weeks in antidepressant studies in the FDA database.

Symptom remission is considered a desired goal in depression treatment because it is associated with better daily functioning and better long-term prognosis. Dextromethorphan-bupropion treatment resulted in early and substantial achievement of remission on the MADRS (total score≤10), with statistically significant separation from placebo demonstrated at week 2 and at every subsequent time point. At week 6, the percentage of patients achieving remission was 39.5% in the dextromethorphan-bupropion group compared to 17.3% in the placebo group (P=0.004). Clinical response on the MADRS (≥50% reduction from baseline) was also early and substantial with statistical significance versus placebo observed at week 1 and at every subsequent time point. At week 6, the percentage of patients achieving clinical response was 54.0% in the dextromethorphan-bupropion group compared to 34.0% in the placebo group (P<0.001).

The early efficacy of dextromethorphan-bupropion was further evidenced by statistically significant benefits versus placebo at week 1 and at all subsequent time points on numerous other clinically relevant measures including MADRS-6, CGI-S, CGI-I, PGI-I, QIDS-SR-16, and Q-LES-Q-SF. Improvement in functional disability was also observed early with statistical significance on the SDS achieved at week 2 and at every time point thereafter.

Dextromethorphan-bupropion was safe and well tolerated in this trial, with low rates of discontinuations due to adverse events. The magnitude of the differences in the rates of adverse events and discontinuations due to adverse events between the two treatment groups in the trial likely reflects the much lower-than-expected rate of these events in the placebo group. For example, the 61.7% overall rate of adverse events in the dextromethorphan-bupropion group is in line with the average of 76.4% reported for antidepressant arms in a large meta-analysis of placebo-controlled depression trials. In comparison, the 45.1% rate of adverse events for placebo in this trial is lower than the 63.0% average reported for the placebo arms in the same meta-analysis. Similarly, the 6.2% rate of discontinuations due to adverse events in the dextromethorphan-bupropion group is in line with the average of 7% reported for antidepressant arms in another large meta-analysis of placebo-controlled depression trials. In contrast, the 0.6% rate of discontinuations due to adverse events for placebo in our trial is substantially lower than the 4% average reported for the placebo arms in that same meta-analysis. Unlike other NMDA receptor antagonists, dextromethorphan-bupropion was not associated with psychotomimetic effects. This tolerability profile could be related to the significantly faster rate of unblocking of the NMDA receptor channel reported for dextromethorphan as compared to other NMDA antagonists. Dextromethorphan-bupropion was not associated with weight gain or increased sexual dysfunction.

Limitations of this trial include exclusion of patients with psychotic or other psychiatric disorders, alcohol/substance use disorders, clinically significant risk of suicide, or significant medical comorbidities. These exclusions along with prohibition of certain concomitant medications may limit the generalizability of the study findings. In addition, treatment at experienced trial sites by specialized clinicians under a trial protocol with frequent clinical assessments may not reflect general practice. The dropout rate of 24.1% in the dextromethorphan-bupropion group is approximately twice that of placebo. However, this rate is in line with the average of 24.3% reported for antidepressant arms in a large meta-analysis of published placebo-controlled depression trials. In contrast, the 10.4% dropout rate for placebo in this trial is substantially lower than the 24.0% average reported for placebo arms in the same meta-analysis. Multiplicity adjustments were only applied to the key secondary efficacy endpoints; however, the nominal P values for most other secondary endpoints were <0.05. Finally, the trial duration was limited to 6 weeks. The effect of long-term treatment with dextromethorphan-bupropion has been evaluated in a separate study.

In conclusion, this study demonstrated that treatment with dextromethorphan-bupropion resulted in clinically meaningful and statistically significant improvements in depressive symptoms compared to placebo starting at week 1 in patients with MDD and was well tolerated. The efficacy of dextromethorphan-bupropion was supported by significant improvements compared to placebo on multiple clinically relevant endpoints across symptom-specific and global measures, demonstrating internal consistency of the study results.

Unless otherwise indicated, all numbers expressing quantities of ingredients, properties such as amounts, percentage, and so forth used in the specification and claims are to be understood in all instances as indicating both the exact values as shown and as being modified by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the embodiments (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the embodiments and does not pose a limitation on the scope of any claim. No language in the specification should be construed as indicating any non-claimed element essential to the practice of the claims.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or to expedite prosecution. When any such inclusion or deletion occurs, the specification is deemed to contain the group as modified thus fulfilling the written description of all Markush groups if used in the appended claims.

Certain embodiments are described herein, including the best mode known to the inventors for carrying out the claimed embodiments. Of course, variations on these described embodiments will become apparent to those of ordinary skill in the art upon reading the foregoing description. The inventor expects skilled artisans to employ such variations as appropriate, and the inventors intend for the claimed embodiments to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A method of treating anxiety in a human patient experiencing major depressive disorder, comprising: selecting a human patient experiencing anxiety and major depressive disorder, and administering, to the human patient, a combination of 45 mg of dextromethorphan hydrobromide, or a molar equivalent amount of another salt of dextromethorphan or the free base form of dextromethorphan, and 105 mg of bupropion hydrochloride, or a molar equivalent amount of another salt of bupropion or the free base form of bupropion.

2. The method of claim 1, wherein the human patient is selected for having a Hamilton Anxiety Rating Scale (HAM-A) score of at least 10 prior to treatment.

3. The method of claim 2, wherein the human patient is selected for having a HAM-A score of at least 15 prior to treatment.

4. The method of claim 1, wherein the human patient's HAM-A score is reduced by about 3 to about 4 after 1 week of treatment.

5. The method of claim 1, wherein the human patient's HAM-A score is reduced by about 5 to about 6 after 2 weeks of treatment.

6. The method of claim 1, wherein the human patient's HAM-A score is reduced by about 8 to about 9 after 6 weeks of treatment.

7. The method of claim 1, wherein the human patient achieves remission of anxiety based upon the human patient's HAM-A score after one or more weeks of treatment.

8. A method of treating anhedonia in a human patient experiencing major depressive disorder, comprising: selecting a human patient experiencing anhedonia and major depressive disorder, and administering, to the human patient, a combination of 45 mg of dextromethorphan hydrobromide, or a molar equivalent amount of another salt of dextromethorphan or the free base form of dextromethorphan, and 105 mg of bupropion hydrochloride, or a molar equivalent amount of another salt of bupropion or the free base form of bupropion.

9. The method of claim 8, wherein the human patient is selected for having a CGI-S score of at least 4 prior to treatment.

10. The method of claim 8, wherein the human patient is selected for having a Montgomery-Åsberg Depression Rating Scale (MADRS) Anhedonia score of at least 15 prior to treatment.

11. The method of claim 8, wherein the human patient is selected for having a MADRS Anhedonia score of at least 19 prior to treatment.

12. The method of claim 8, wherein the human patient's MADRS Anhedonia score is reduced by about 4 to about 5 after 1 week of treatment.

13. The method of claim 8, wherein the human patient's MADRS Anhedonia score is reduced by about 9 to about 10 after 6 weeks of treatment.

14. The method of claim 1, wherein the combination is administered twice a day.

15. The method of claim 1, wherein the human patient is selected for having a MADRS score of at least 25 prior to treatment.

16. The method of claim 8, wherein the combination is administered twice a day.

17. The method of claim 8, wherein the human patient is selected for having a MADRS score of at least 25 prior to treatment.

18. The method of claim 1, wherein the combination comprises 45 mg of dextromethorphan hydrobromide and 105 mg of bupropion hydrochloride.

19. The method of claim 8, wherein the combination comprises 45 mg of dextromethorphan hydrobromide and 105 mg of bupropion hydrochloride.

20. The method of claim 1, wherein the combination is orally administered to the human patient.

21. The method of claim 8, wherein the combination is orally administered to the human patient.
